(19) United States
(12) Patent Application Publication
(10) Pub. No.: US 2018/0263940 A1
(43) Pub. Date: Sep. 20, 2018

(54) TREATMENT OF SKIN CONDITIONS

(71) Applicant: MONASH UNIVERSITY, Clayton, Victoria (AU)

(72) Inventors: Ian SMYTH, Clayton, Victoria (AU); Denny COTTLE, Clayton, Victoria (AU); Gloria URSINO, Clayton, Victoria (AU)

(21) Appl. No.: 15/557,405

(22) PCT Filed: Mar. 16, 2016
(86) PCT No.: PCT/AU2016/050185

§ 371 Date: Sep. 11, 2017

(30) Foreign Application Priority Data
Mar. 16, 2015 (AU) 2015900943

(51) Int. Cl. A61K 31/196; A61K 9/00; A61P 17/06; A61P 17/16; A61K 47/44

(52) U.S. Cl. CPC A61K 31/196; A61K 9/0014; A61P 17/06; A61K 31/202; A61K 47/44; A61K 2300/00; A61P 17/16

(57) ABSTRACT

The present invention relates to compositions, methods and kits for the treatment of dermopathies. In particular, the compositions, methods and kits are particularly useful, but not limited to, the treatment of ichthyoses such as Harlequin Ichthyosis. The present invention provides a method of treating a skin condition associated with lipid dysfunction, the method comprising administering to a subject in need thereof aminosalicyclic acid (ASA), ASA derivative, or pharmaceutically acceptable salt, ester, amide, polymorph and/or prodrug thereof, thereby treating a skin condition associated with lipid dysfunction. Preferably, the skin condition associated with lipid dysfunction is an ichthyoses, for example Harlequin Ichthyosis. Preferably, the ASA is mesalamine.

### CROSS REFERENCE(S) TO RELATED APPLICATIONS

This application claims priority from Australian provisional application no. 2015900943, the entire contents of which are hereby incorporated by reference.

### FIELD OF THE INVENTION

The present invention relates to compositions, methods and kits for the treatment of dermopathies. In particular, the compositions, methods and kits are particularly useful, but not limited to, the treatment of ichthyoses such as Harlequin Ichthyosis.

### BACKGROUND OF THE INVENTION

The epidermis is a stratified epithelium which creates a barrier to dehydration, the environment and infection. Befitting the central role that the organ plays in defence of the body against the external environment, approximately 15% of doctor consultations concern skin conditions and the associated costs to the health system are therefore significant. Defects in the skin are also remarkably numerous, with over 4000 different dermopathies having been described.

The ichthyoses are a family of at least 20 congenital diseases characterized by the development of a thick hyperkeratotic epidermis. The most severe form of this disease spectrum is Harlequin Ichthyosis (HI) (OMIM #242500) which is caused by mutations in ABCA12, a putative lipid transport protein of the ATP binding cassette (ABC) family. HI is a rare but very severe skin disease, with ˜50% neonatal lethality, although their disease upon delivery is already extreme. For those patients who do survive beyond birth, a modest improvement in disease phenotypes is observed, although a lifetime regime of frequent bathing, removal of scales and frequent application of emollient oils is required to manage the disorder. In mouse models of HI neonatal mortality is fully penetrant, but grafted fetal skin exhibits an analogous self-improvement which has been attributed to better keratinocyte differentiation. Retinoid therapy is the main treatment for HI neonates, as they typically promote keratinocyte differentiation and shedding. However, there are a number of undesirable side-effects that limit their long-term use. Their effectiveness as an HI treatment is also under review amid debate about whether the perceived improvement in disease is a function of retinoid activity or the product of better disease management.

There exists a need for new or improved treatments for dermopathies, particularly ichthyoses such as Harlequin Ichthyosis.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a method of treating a skin condition associated with lipid dysfunction, the method comprising administering to a subject in need thereof aminosalicyclic acid (ASA) having the structure:

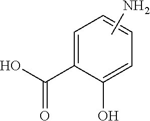

or ASA derivative, pharmaceutically acceptable salt, ester, amide, polymorph and/or prodrug thereof, thereby treating a skin condition associated with lipid dysfunction. Preferably, the ASA is 5-aminosalicylic acid (5-ASA: also known as mesalamine or mesalazine), 4-ASA or 3-ASA, derivative, pharmaceutically acceptable salt, ester, amide, polymorph and/or prodrug thereof. Even more preferably the compound is mesalamine, derivative, pharmaceutically acceptable salt, ester, amide, polymorph and/or prodrug thereof.

Preferably the skin condition associated with a lipid dysfunction is an ichthyosis. Preferably, the ichthyosis is selected from the group consisting of Harlequin Ichthyosis, Lamellar Ichthyosis including various subtypes such as Lamellar Ichthyosis Type 1, 2 or 3, Congenital Ichthyosiform Erythroderma types, Acral Peeling Skin Syndrome, Netherton Syndrome, Chanarin-Dorfman syndrome (Neutral lipid storage disease with Ichthyosis), X-linked Ichthyosis, Arthrogryposis-renal dysfunction-cholestasis (ARC) syndrome, Ichthyosis Vulgaris, Niemann-Pick Disease, Gaucher's Disease and HXALI hepoxilin A3 synthase-linked ichthyosis.

Preferably the ichthyoses is selected from the group consisting of Harlequin Ichthyosis, Lamellar Ichthyosis including various subtypes such as Lamellar Ichthyosis Type 1, 2 or 3, Congenital Ichthyosiform Erythroderma types, Chanarin-Dorfman syndrome (Neutral lipid storage disease with Ichthyosis), X-linked Ichthyosis, Niemann-Pick Disease, Gaucher's Disease, HXALI hepoxilin A3 synthase-linked ichthyosis.

Even more preferably, the ichthyoses is Harlequin Ichthyosis or Lamellar Ichthyosis.

In one aspect, the present invention provides a method of treating a skin condition associated with lipid dysfunction, the method comprising administering to a subject in need thereof ASA, ASA derivative, or pharmaceutically acceptable salt, ester, amide, polymorph and/or prodrug thereof, thereby treating a skin condition associated with lipid dysfunction. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

In one aspect, the invention also provides a method of alleviating or ameliorating a symptom of a skin condition associated with lipid dysfunction, the method comprising administering to a subject in need thereof ASA, ASA derivative, or pharmaceutically acceptable salt, ester, amide, polymorph and/or prodrug thereof, alleviating or ameliorating a symptom of a skin condition associated with lipid dysfunction. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

In another aspect, the invention also provides use of ASA, ASA derivative, or pharmaceutically acceptable salt, ester, amide, polymorph and/or prodrug thereof in the manufacture of a medicament for the treatment of a skin condition associated with lipid dysfunction. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

In any method or use of the invention described herein, the ASA, ASA derivative, or pharmaceutically acceptable salt, ester, amide, polymorph and/or prodrug thereof may be administered directly to the skin. Preferably, the administration to the skin is via any route that allows ASA, ASA derivative, or pharmaceutically acceptable salt, ester, amide, polymorph and/or prodrug thereof to contact the epidermis or a part thereof. For example, the ASA, ASA derivative, or pharmaceutically acceptable salt, ester, amide, polymorph and/or prodrug thereof may be administered via any route such that it contacts any one of the layers that comprise the epidermis such as the basal layer, spinous layer, granular layer and stratum corneum. Preferably, the ASA, ASA derivative, or pharmaceutically acceptable salt, ester, amide, polymorph and/or prodrug thereof is applied to the skin topically.

In one aspect, the method for the treatment of a skin condition associated with lipid dysfunction comprises the steps of

identifying a subject having a skin condition associated with lipid dysfunction; and

administering to the subject in need thereof ASA, ASA derivative, or pharmaceutically acceptable salt, ester, amide, polymorph and/or prodrug thereof,

thereby treating a skin condition associated with lipid dysfunction. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

In one aspect, the present invention provides a method for the treatment of Harlequin Ichthyosis comprising the steps of

identifying a subject having Harlequin Ichthyosis; and

administering to the subject in need thereof ASA, ASA derivative, or pharmaceutically acceptable salt, ester, amide, polymorph and/or prodrug thereof,

thereby treating Harlequin Ichthyosis. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

In another aspect, the present invention provides a method for the treatment of a subject having Harlequin Ichthyosis comprising the steps of

identifying a subject having Harlequin Ichthyosis, the subject having been treated with retinoid therapy; and

administering to the subject in need thereof ASA, ASA derivative, or pharmaceutically acceptable salt, ester, amide, polymorph and/or prodrug thereof,

thereby treating Harlequin Ichthyosis. Preferably, the subject did not experience any improvement in their condition as a result of retinoid therapy. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

The present invention provides a method for the treatment of a skin condition associated with lipid dysfunction comprising the steps of administering to a subject in need thereof ASA, ASA derivative, or pharmaceutically acceptable salt, ester, amide, polymorph and/or prodrug thereof, and a compound for increasing the barrier function of the skin. Preferably, a compound for increasing the barrier function of the skin creates an artificial skin barrier. Typically, the compound is an oil or lipid emollient. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

The present invention provides a method for the treatment of a skin condition associated with lipid dysfunction comprising the steps of administering a first composition comprising ASA, ASA derivative, or pharmaceutically acceptable salt, ester, amide, polymorph and/or prodrug thereof and a second composition comprising a compound for increasing the barrier function of the skin. Preferably, a compound for increasing the barrier function of the skin creates an artificial skin barrier. Typically, the compound is an oil or lipid emollient. The first and second compositions may be administered sequentially or simultaneously. Preferably, the first composition is administered to the subject prior to the second composition. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

The present invention provides a method for the treatment of a skin condition associated with lipid dysfunction, the method comprising the step of applying a composition topically to the skin of a subject having a skin condition associated with lipid dysfunction, the composition applied in an amount sufficient to cover the area of skin impacted by a skin condition associated with lipid dysfunction; wherein the composition comprises, consists essentially of or consists of ASA, ASA derivative, or pharmaceutically acceptable salt, ester, amide, polymorph and/or prodrug thereof and a pharmaceutically acceptable diluent, excipient or carrier. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

The invention provides a pharmaceutical composition for treating a skin condition associated with lipid dysfunction comprising ASA, ASA derivative, or pharmaceutically acceptable salt, ester, amide, polymorph and/or prodrug thereof and a pharmaceutically acceptable diluent, excipient or carrier. In one embodiment, the only active ingredient present in the composition is ASA, ASA derivative, or pharmaceutically acceptable salt, ester, amide, polymorph and/or prodrug thereof. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

The invention provides a pharmaceutical composition for treating a skin condition associated with lipid dysfunction comprising as active ingredients ASA, ASA derivative, or pharmaceutically acceptable salt, ester, amide, polymorph and/or prodrug thereof and a pharmaceutically acceptable diluent, excipient or carrier. In one embodiment, the only active ingredient present in the composition is ASA, ASA derivative, or pharmaceutically acceptable salt, ester, amide, polymorph and/or prodrug thereof. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

The invention provides a pharmaceutical composition for treating a skin condition associated with lipid dysfunction comprising as main ingredients ASA, ASA derivative, or pharmaceutically acceptable salt, ester, amide, polymorph and/or prodrug thereof and a pharmaceutically acceptable diluent, excipient or carrier. In one embodiment, the only active ingredient present in the composition is ASA, ASA derivative, or pharmaceutically acceptable salt, ester, amide, polymorph and/or prodrug thereof. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

The invention also provides ASA, ASA derivative, or pharmaceutically acceptable salt, ester, amide, polymorph and/or prodrug thereof for use in the treatment of a skin condition associated with lipid dysfunction. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

The invention also provides a pharmaceutical composition comprising ASA, ASA derivative, or pharmaceutically acceptable salt, ester, amide, polymorph and/or prodrug thereof and a pharmaceutically acceptable diluent, excipient or carrier for use in the treatment of a skin condition associated with lipid dysfunction. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

The present invention also provides a cosmetic composition for improving the appearance of the skin comprising ASA, ASA derivative, or pharmaceutically acceptable salt, ester, amide, polymorph and/or prodrug thereof and a cosmetically acceptable diluent, excipient or carrier. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

Any pharmaceutical or cosmetic composition of the invention may comprise one or more ASAs, ASA derivatives, or pharmaceutically acceptable salts, esters, amides, polymorphs and/or prodrugs thereof. For example, the composition may include 5-ASA and 4-ASA.

Any pharmaceutical or cosmetic composition or method of the invention may further comprise a retinoid or administration of a retinoid. Preferably, the retinoid is one that is used for treating an ichthyosis, preferably, HI. Preferably, the retinoid is acitretin, etretinate, isotretinoin or tazarotene. The retinoid may be administered systemically or topically, for example tazarotene may be administered topically. Typically, the retinoid is present in the composition at a dose lower than when it is used as a monotherapy or when used in a therapy as the only active ingredient.

Any pharmaceutical or cosmetic composition of the invention for topical administration may be formulated as a lotion, cream, oil, a stick- or bar-shaped solid, a spray, an ointment, a paste, mousse, a body wash, or a cosmetic.

The present invention also provides a method for improving the appearance of the skin in a subject having a skin condition associated with lipid dysfunction, the method comprising administering ASA, ASA derivative, or pharmaceutically acceptable salt, ester, amide, polymorph and/or prodrug thereof or a cosmetic composition described herein. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

The invention provides a kit or article of manufacture including ASA, ASA derivative, or pharmaceutically acceptable salt, ester, amide, polymorph and/or prodrug thereof, and/or a pharmaceutical composition described herein.

The present methods, uses and compositions can also be used to treat skin conditions associated with dysregulated or unregulated proliferation, differentiation or migration of keratinocytes in the epidermis. For example, the present invention also provides a method of treating a skin condition associated with dysregulated or unregulated proliferation, differentiation or migration of keratinocytes in the epidermis, the method comprising administering to a subject in need thereof aminosalicyclic acid (ASA) having the structure:

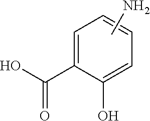

or ASA derivative, pharmaceutically acceptable salt, ester, amide, polymorph and/or prodrug thereof, thereby treating a skin condition associated with dysregulated or unregulated proliferation, differentiation or migration of keratinocytes in the epidermis. Preferably, the ASA is 5-aminosalicylic acid (5-ASA: also known as mesalamine or mesalazine), 4-ASA or 3-ASA, derivative, pharmaceutically acceptable salt, ester, amide, polymorph and/or prodrug thereof. Even more preferably the compound is mesalamine, derivative, pharmaceutically acceptable salt, ester, amide, polymorph and/or prodrug thereof. Examples of skin conditions associated with dysregulated or unregulated proliferation, differentiation or migration of keratinocytes in the epidermis are described herein.

The present methods, uses and compositions can also be used to treat skin conditions associated with parakeratosis. For example, the present invention also provides a method of treating a skin condition associated with parakeratosis, the method comprising administering to a subject in need thereof aminosalicyclic acid (ASA) having the structure:

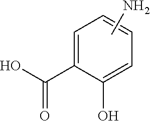

or ASA derivative, pharmaceutically acceptable salt, ester, amide, polymorph and/or prodrug thereof, thereby treating a skin condition associated with parakeratosis. Preferably, the ASA is 5-aminosalicylic acid (5-ASA: also known as mesalamine or mesalazine), 4-ASA or 3-ASA, derivative, pharmaceutically acceptable salt, ester, amide, polymorph and/or prodrug thereof. Even more preferably the compound is mesalamine, derivative, pharmaceutically acceptable salt, ester, amide, polymorph and/or prodrug thereof. Examples of skin conditions associated with parakeratosis are described herein.

The present methods, uses and compositions can also be used to treat skin conditions associated with loss of the granular layer in the epidermis. For example, the present invention also provides a method of treating a skin condition associated with loss of the granular layer in the epidermis, the method comprising administering to a subject in need thereof aminosalicyclic acid (ASA) having the structure:

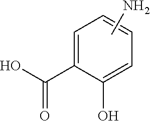

or ASA derivative, pharmaceutically acceptable salt, ester, amide, polymorph and/or prodrug thereof, thereby treating a skin condition associated with loss of the granular layer in the epidermis. Preferably, the ASA is 5-aminosalicylic acid (5-ASA: also known as mesalamine or mesalazine), 4-ASA or 3-ASA, derivative, pharmaceutically acceptable salt, ester, amide, polymorph and/or prodrug thereof. Even more preferably the compound is mesalamine, derivative, pharmaceutically acceptable salt, ester, amide, polymorph and/or prodrug thereof. Examples of skin conditions associated with loss of the granular layer in the epidermis are described herein.

The present methods, uses and compositions can also be used to treat skin conditions associated with hyperkeratotic epidermis. For example, the present invention also provides a method of treating a skin condition associated with hyperkeratotic epidermis, the method comprising administering to a subject in need thereof aminosalicyclic acid (ASA) having the structure:

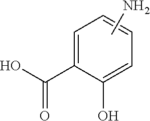

or ASA derivative, pharmaceutically acceptable salt, ester, amide, polymorph and/or prodrug thereof, thereby treating a skin condition associated with hyperkeratotic epidermis. Preferably, the ASA is 5-aminosalicylic acid (5-ASA: also known as mesalamine or mesalazine), 4-ASA or 3-ASA, derivative, pharmaceutically acceptable salt, ester, amide, polymorph and/or prodrug thereof. Even more preferably the compound is mesalamine, derivative, pharmaceutically acceptable salt, ester, amide, polymorph and/or prodrug thereof. Examples of skin conditions associated with hyperkeratotic epidermis are described herein.

The present invention provides a method of treating an ichthyosis in a subject in need thereof, the method comprising topically applying to the skin a composition comprising mesalamine, thereby treating an ichthyosis. Preferably, the composition further comprises a retinoid such as any described herein.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Overview of normal keratinocyte differentiation. The epidermis portion of skin is composed of 4 distinct layers and keratinocytes progressively move upwards through these phases starting at the basal layer (where proliferation normally occurs) until being shed as a dead husk at the top of the stratum corneum. Each phase has a particular biochemical identity.

FIG. 2: Mesalamine treatment during Harlequin Ichthyosis (HI) disease acquisition—Morphology. Haematoxylin and Eosin staining of E16.5 embryonic mouse dorsal skin cultured ex vivo for 4 days on chamber inserts, with 10 mM mesalamine in standard culture media or vehicle alone. +/+ presents wild type skin. Lx12/Lx12 represents the HI mutant skin and Lx12/+ represents seemingly normal skin carrying the recessive HI mutant allele. Note the abnormality observed in the spinous layer, loss of the granular layer and thickening of the stratum corneum in Lx12/Lx12 (HI) vehicle treated skin. However upon mesalamine treatment the HI epidermis acquires a more-normal appearance. Mesalamine did not however appear to greatly effect+/+ skin. Images are repeated in grayscale.

FIG. 3: Mesalamine treatment during Harlequin Ichthyosis (HI) disease acquisition—Apoptosis. Immunofluorescent staining of E16.5 embryonic mouse dorsal skin cultured ex vivo for 4 days on chamber inserts, with 10 mM mesalamine in standard culture media or vehicle alone. +/+ presents wild type skin. Lx12/Lx12 represents the HI mutant skin and Lx12/+ represents seemingly normal skin carrying the recessive HI mutant allele. A) In Red the Keratin 14 (K14) basal epidermal marker is observed, in Blue is the nuclear dye, DAPI and in Green is the marker of apoptosis (programed cell death), cleaved-Caspase-3 (cleaved-Casp3). The images are also repeated with focus only on cleaved-Caspase-3 in greyscale. Dashed line indicates boundary between epidermis and dermis. B) Quantification of the number of apoptotic (cleaved-Caspase-3 positive) cells observed, across the skin section length for four siblings of +/+, Lx12/+ and Lx12/Lx12 (x2) genotypes. Note: The epidermis is normally very resistant to apoptosis, however there are an increasing number of apoptotic keratinocytes observed in Lx12/+ and Lx12/Lx12 (HI) vehicle treated skin. Upon mesalamine treatment however, apoptosis is reduced.

FIG. 4: Mesalamine treatment during Harlequin Ichthyosis (HI) disease acquisition—Keratin 10—differentiation. Immunofluorescent staining of E16.5 embryonic mouse dorsal skin cultured ex vivo for 4 days on chamber inserts, with 10 mM mesalamine in standard culture media or vehicle alone. +/+ presents wild type skin. Lx12/Lx12 represents the HI mutant skin and Lx12/+ represents seemingly normal skin carrying the recessive HI mutant allele. In Red the Keratin 14 (K14) basal epidermal marker is observed, in Blue is the nuclear dye, DAPI and in Green is the spinous layer marker, Keratin 10 (K10). The images are also repeated with focus only on K10 in greyscale. Dashed line indicates boundary between epidermis and dermis. Note the decreasing number of K10+ve keratinocytes observed in both Lx12/+ and Lx12/Lx12 (HI) vehicle treated skin. Upon mesalamine treatment however, K10 cells revert to normal levels in Lx12/+ skin, and some modest improvement is seen in Lx12/Lx12 skin.

FIG. 5: Mesalamine treatment during Harlequin Ichthyosis (HI) disease acquisition—Involucrin—differentiation. Immunofluorescent staining of E16.5 embryonic mouse dorsal skin cultured ex vivo for 4 days on chamber inserts, with 10 mM mesalamine in standard culture media or vehicle alone. +/+ presents wild type skin. Lx12/Lx12 represents the HI mutant skin and Lx12/+ represents seemingly normal skin carrying the recessive HI mutant allele. In Red the Keratin 14 (K14) epidermal marker is observed, in Blue is the nuclear dye, DAPI and in Green is the spinous and granular layer marker, Involucrin (INV). The images are also repeated with focus only on INV in greyscale. Dashed line indicates boundary between epidermis and dermis. Brackets indicate predominant expression of INV. Note: INV is most strongly detected in the granular layers but exhibits disorganised and premature expression in Lx12/Lx12 (HI) vehicle treated skin. Upon mesalamine treatment however, a more organised, and delayed (granular layer) expression of INV is seen in Lx12/Lx12 skin.

FIG. 6: Mesalamine treatment during Harlequin Ichthyosis (HI) disease acquisition—Loricrin—differentiation. Immunofluorescent staining of E16.5 embryonic mouse dorsal skin cultured ex vivo for 4 days on chamber inserts, with 10 mM mesalamine in standard culture media or vehicle alone. +/+ presents wild type skin. Lx12/Lx12 represents the HI mutant skin and Lx12/+ represents seemingly normal skin carrying the recessive HI mutant allele. In Red the Keratin 14 (K14) epidermal marker is observed, in Blue is the nuclear dye, DAPI and in Green is the granular layer marker, Loricrin (LOR). The images are also repeated with focus only on LOR in greyscale. Dashed line indicates boundary between epidermis and dermis. Brackets indicate predominant expression of LOR. Note: LOR is most strongly detected in the granular layers and to lesser extent in the stratum corneum. Premature expression of LOR in basal and suprabasal keratinocytes of Lx12/Lx12 (HI) vehicle treated skin is observed, however, upon mesalamine treatment a more normal, predominantly granular layer expression of LOR is seen in Lx12/Lx12 skin.

FIG. 7: Mesalamine treatment during Harlequin Ichthyosis (HI) disease acquisition—Filaggrin—differentiation. Immunofluorescent staining of E16.5 embryonic mouse dorsal skin cultured ex vivo for 4 days on chamber inserts, with 10 mM mesalamine in standard culture media or vehicle alone. +/+ presents wild type skin. Lx12/Lx12 represents the HI mutant skin and Lx12/+ represents seemingly normal skin carrying the recessive HI mutant allele. In Red the Keratin 14 (K14) epidermal marker is observed, in Blue is the nuclear dye, DAPI and in Green is the granular to cornified layer marker, Filaggrin (FLG). The images are also repeated with focus only on FLG in greyscale. Dashed line indicates boundary between epidermis and dermis. Brackets indicate predominant expression of FLG. Note: Filaggrin is most strongly detected in the granular layers and to lesser extent in the stratum corneum. Progressive loss of granular layer FLG in both Lx12/+ and Lx12/Lx12 (HI) vehicle treated skin, is observed, however, upon mesalamine treatment granular layer FLG is restored to Lx12/+ skin and partially restored to Lx12/Lx12 skin.

FIG. 8: The effect of altered dosages of mesalamine treatment during Harlequin Ichthyosis (HI) disease acquisition—Morphology. Haematoxylin and Eosin staining of embryonic mouse dorsal skin from a second E16.5 litter, cultured ex vivo for 4 days on chamber inserts, with 1 or 10 mM mesalamine in standard culture media or vehicle alone. +/+ presents wild type skin. Lx12/Lx12 represents the HI mutant skin and Lx12/+ represents seemingly normal skin carrying the recessive HI mutant allele. Note the abnormality observed in the spinous layer, loss of the granular layer and proportional thickening of the stratum corneum in Lx12/Lx12 (HI) vehicle treated skin. However upon mesalamine treatment the epidermis acquires a more-normal appearance. Representative images are provided at two magnifications to highlight the uniformity of the effects across the skin, and repeated in grayscale.

FIG. 9: Mesalamine treatment after Harlequin Ichthyosis (HI) disease acquisition—Morphology. Representative Haematoxylin and Eosin staining of embryonic mouse dorsal skin from two E18.5 litters, cultured ex vivo for 4 days on chamber inserts, with 1 or 10 mM mesalamine in standard culture media or vehicle alone. +/+ presents wild type skin. Lx12/Lx12 represents the HI mutant skin and Lx12/+ represents seemingly normal skin carrying the recessive HI mutant allele. Note once again the abnormality observed in the spinous layer, loss of the granular layer and proportional thickening of the stratum corneum in Lx12/Lx12 (HI) vehicle treated skins. Lx12/+ skins also appeared slightly abnormal relative to +/+ vehicle treated skins. However upon 1 mM mesalamine treatment, the HI epidermis acquires a more-normal appearance again, while 10 mM mesalamine in this context appears to affect all genotypes by reducing the keratinocytes accumulating in the spinous layers (indicative of enhanced terminal differentiation). This suggests the optimal dose will be lower than 10 mM, and likely close to 1 mM. Images are repeated in grayscale.

FIG. 10: Mesalamine is able to correct Cornified Layer thickening in Grainyhead-like 3 (GRHL3) −/− epidermis (a proxy model of Lamellar Ichthyosis). A) Haematoxylin and Eosin stained GRHL3+/+(Wild type) and GRHL3 −/− (knockout) skin collected at E16.5 and cultured for 4 days in ex vivo whole mount assay, with and without 10 mM Mesalamine. Media was refreshed at 48 hrs. Note hyper-thickening of cornified layer marked by bracket and CL in vehicle-treated GRHL3 −/− skins relative to GRHL3+/+ skins and loss of thickening upon 10 mM Mesalamine treatment. B) Quantification of Cornified layer thickness. n=4 mice per condition, with phenotypically normal GRHL3+/+ and GRHL3+/− skins pooled as a control group. P values from student's t test.

FIG. 11: Creation of conditional inducible adult mouse model of Harlequin Ichthyosis. (A) Mice of the Abca12 tm1c/tm1c K14-CreER genotype develop darkened, dry, wrinkled and inflexible back skin and (B) scaling and cracking of the throat skin by 11 days after tamoxifen (4-hydroxytamoxifen (4OHT)) exposure as compared to control mice lacking the Cre transgene. (C) At a histological level the skin of Abca12 tm1c/tm1c K14-CreER+4OHT mice show thickening of the epidermis (especially the cornified cell layer), wounds and dermal immune infiltrate consistent with many of the features of human Harlequin Ichthyosis. (D) PCR analysis of skin biopsies confirms the generation of the deleted Abca12 tm1d allele only in mice carrying at least 1 Abca12 tm1c gene, that also carry the K14-CreER transgene and where exposed to 4OHT.

FIG. 12: Topical application of Mesalamine promotes orthokeratosis in mouse tail scale assay. (A) Phenotypically normal mice had Mesalamine Cream (or Base cream alone) applied twice daily to the naturally parakeratotic tail scale epidermis for 6 days. Haematoxylin and Eosin staining was performed on paraffin tissues sections prepared from the harvested tail skin. (B) The total scale length was measured as indicated by line B and the length of the granular layer was measured as indicated by line A in (A). The % of the scale showing a granular layer (visible as dark line under cornified envelope), also known as % orthokeratosis, was quantified by dividing the length of line A by the total length of the scale line B×100% from each condition. N=3 mice per condition. P value from student's t test.

FIG. 13: Additive action of Mesalamine and Acitretin increases orthokeratosis in Ex vivo mouse tail scale assay. (A) Naturally parakeratotic tail scale skin was collected from wild type mice and cultured for 4 days in ex vivo whole mount assay, with and without 2 and 5 mM Mesalamine and/or 1 μM Acitretin. (A) Haematoxylin and Eosin staining was performed on paraffin tissues sections prepared from the cultured tail skin. (B) The total scale length was measured as indicated by line B and the length of the granular layer was measured as indicated by line A in (A). The % of the scale showing a granular layer (visible as very dark line under cornified envelope), also known as % orthokeratosis, was quantified by dividing the length of line A by the total length of the scale line B×100% from each condition. N=3 mice per condition. P values are from pairwise comparisons of key conditions using the student's t test.

FIG. 14: Mesalamine treatment is more effective than Acitretin in lowering Cornified Envelope thickening in Harlequin Ichthyosis ex vivo embryo whole mount skin assays. Ex vivo embryo whole mount assays where performed with back skin isolated from E16.5 embryos. Skins were cultured without drugs (Vehicle), with 1 μM Acitretin or 10 mM Mesalamine. Heterozygous siblings Lx12/+ where used as controls. n=3-8 mice per condition. P values from student's t test.

FIG. 15: Positive changes in epidermal differentiation detected upon first trial of Mesalamine topical cream on live Harlequin Ichthyosis mice. (A) Abca12 tm1c/tm1c K14CreER+4OHT treated mice had 2% Mesalamine Cream (or Base cream alone) applied twice daily to the lower back epidermis for 6 days, following induction of Harlequin Ichthyosis with 4OHT for 5 days. Haematoxylin and Eosin staining was performed on paraffin tissues sections prepared from the harvested skin. Gross skin morphology was similar between each test condition however the number of scabs (from cracking wounds) evident in the cornified layer (indicated by black arc lines) was approximately halved upon addition of Mesalamine (B) and analysis of the composition of epidermal thickness showed that the cornified layer had reduced by approximately 5% while the nucleated cell layers (basal and spinous layers) had gained approximately 5% (C). D) Tissue from these mice was further stained for the spinous layer marker Keratin 10 (K10). Black arc lines indicate patches of K10 expression. Upon induction of Harlequin Ichthyosis K10 expression was almost entirely abolished, however partial reacquisition of K10 was detected across some mice treated with the base cream. Upon treatment with the Mesalamine cream however, all mice consistently reacquired K10 expression throughout the majority of the epidermis. (E) Blood serum was harvested from treated mice and a modified Trinder reaction performed to assess serum Salicylate levels. Mice treated with topical Mesalamine cream did not show increased serum Salicylate relative to base cream treated mice nor was the level above the toxic threshold of 35 mg/dl. F) Mice treated with topical Mesalamine cream did however show elevated urine Salicylate using the Trinder method as expected and indicating any systemic Mesalamine is being efficiently cleared from the body via urination.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The inventors have identified a novel treatment for a range of skin conditions associated with lipid dysfunction. In particular, the treatment is useful for conditions in which the epidermis of the skin is defective in extracellular lipid trafficking, has increased intracellular lipid accumulation, typically within keratinocytes, and/or has a reduction in extracellular lipid lamellae compared to normal healthy epidermis of the skin. The treatment involves application of ASA, ASA derivative, or pharmaceutically acceptable salt, ester, amide, polymorph and/or prodrug thereof. The present treatment for skin conditions associated with lipid dysfunction, such as the ichthyoses, are limited. Specifically, for the rare Harlequin Ichthyosis (HI) retinoid therapy is the main treatment for neonates who survive beyond birth, however, there are a number of undesirable side-effects that limit long term use and its effectiveness is debated. While a modest improvement in disease phenotypes is observed in survivors, a lifetime regime of frequent bathing, removal of scales and frequent application of emollient oils is required to manage the disorder. The present invention has the advantage of rescuing skin differentiation defects including correcting keratinocyte differentiation.

A ‘skin condition associated with lipid dysfunction’ includes a condition of the epidermis of the skin in which there is defective extracellular lipid trafficking, increased intracellular lipid accumulation, typically within keratinocytes, and/or reduction in extracellular lipid lamellae compared to normal healthy epidermis of the skin. Whether a skin condition is associated with lipid dysfunction will be understood by a person skilled in the art or can be determined using clinical or biochemical techniques, including but not limited to, those described herein. Preferably, a skin condition associated with lipid dysfunction is an ichthyoses. The ichthyoses may be syndromic or non-syndromic. Non-limiting examples of ichthyoses include Harlequin Ichthyosis, Lamellar Ichthyosis including various subtypes such as Lamellar Ichthyosis Type 1, 2 or 3, Congenital Ichthyosiform Erythroderma types, Acral Peeling Skin Syndrome, Netherton Syndrome, Chanarin-Dorfman syndrome (Neutral lipid storage disease with Ichthyosis), X-linked Ichthyosis, Arthrogryposis-renal dysfunction-cholestasis (ARC) syndrome, Ichthyosis Vulgaris, Niemann-Pick Disease, Gaucher's Disease, autosomal recessive congenital ichthyosis 2 (ARCI2), autosomal recessive congenital ichthyosis 2 (ARCI3), autosomal recessive congenital ichthyosis 8, ichthyosis prematurity syndrome and HXALI hepoxilin A3 synthase-linked ichthyosis. The autosomal recessive congenital ichthyosis and/or skin conditions associated with lipid dysfunction may be caused by a defect, such as a mutation, in any one of the following genes ABCA12, TGM1, TGM5, NIPA1, NIPA2, NIPAL2, NIPAL4, SLC27A4 (FATP4), ALOX12B, ALOXE3, CYP4F22, CYP4V2, PNPLA1, LIPN, CERS3 (Lass3), SPINK5, ABHD5 (CGI-58), STS, Nfe2I2 (Nrf2/Keap 1), VPS33B, FLG, aSMase (smpd1), Beta-glucocerebrosidase (GBA) and Hepoxilin A3 (HXA3) synthase. The skin condition may also be heterozygous carriers of a genetic defect from the genes listed above who do not manifest an ichthyosis but may have an increased incidence of eczema-like type sensitivities. The skin condition may also be heterozygous carriers of a genetic defect in the Abca12 gene who do not manifest HI but may have an increased incidence of eczema-like type sensitivities.

The skin condition associated with lipid dysfunction may also include a condition characterised by a defect, such as a mutation, in any one of the following genes Abca12, TGM1, TGM5, NIPA1, NIPA2, NIPAL2, NIPAL4, SLC27A4 (FATP4), ALOX12B, ALOXE3, CYP4F22, CYP4V2, PNPLA1, LIPN, CERS3 (Lass3), SPINK5, ABHD5 (CGI-58), STS, Nfe2I2 (Nrf2/Keap 1), VPS33B, FLG, aSMase (smpd1), Beta-glucocerebrosidase (GBA) and Hepoxilin A3 (HXA3) synthase.

Other examples of conditions and genes which contain a detect, such as a mutation, are those described in Takeichi et al. Journal of Dermatology 2016; 43: 242-251 and Yoneda, Journal of Dermatology 2016; 43: 252-263, the entire contents of which are incorporated by reference.

Ichthyoses may be diagnosed by clinical and biochemical parameters such as those described in Dermatology, Bolognia, J. L. et al. Saunders; 3 edition (8 Jun. 2012). For example, anosmia may be indicative of X-linked ichthyosis, ataxia and/or cataract may be indicative of neutral lipid storage disease with ichthyosis, bullae/blisters may indicate Congenital Ichthyosiform Erythroderma types, erythroderma may be associated with Congenital Ichthyosiform Erythroderma types, HI, Netherton Syndrome or Lamellar Ichthyosis.

The present methods, uses and compositions can also be used to treat skin conditions associated with dysregulated or unregulated proliferation, differentiation or migration of keratinocytes in the epidermis.

The present methods, uses and compositions can also be used to treat skin conditions associated with parakeratosis.

The present methods, uses and compositions can also be used to treat skin conditions associated with loss of the granular layer in the epidermis.

The present methods, uses and compositions can also be used to treat skin conditions associated with hyperkeratotic epidermis.

The present methods, uses and compositions can also be used to treat conditions such as dermatitis or psoriasis. Preferably, the dermatitis is atopic dermatitis.

Mesalamine is also known as mesalazine or 5-aminosalicylic acid (5-ASA) and has the following structure:

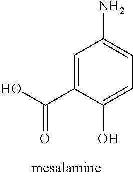 [SMILES: Nc1ccc(O)c(C(=O)O)c1]

Unless the context requires otherwise, use herein of the term ASA includes reference to 3-ASA, 4-ASA, 5-ASA and 6-ASA, and any ASA derivative, or pharmaceutically acceptable salt, ester, amide, polymorph and/or prodrug thereof.

In a similar sense, unless the context requires otherwise, use herein of the term mesalamine includes reference to mesalamine, mesalamine derivative, or pharmaceutically acceptable salt, ester, amide, polymorph and/or prodrug thereof.

The term “pharmaceutically-acceptable salts” refers to those salts which, within the scope of sound medical judgement, are suitable for use in contact with the tissues of humans and animals without undue toxicity, irritation, allergic response and the like, and are commensurate with a reasonable benefit/risk ratio. Pharmaceutically acceptable salts are well known in the art. S. M. Berge et al. describe pharmaceutically acceptable salts in detail in J. Pharmaceutical Sciences, 1977, 66:1-19. The salts include relatively non-toxic, inorganic and organic acid salts of ASA. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, ascorbic, glucoronic, fumaric, maleic, pyruvic, alkyl sulfonic, arylsulfonic, aspartic, glutamic, benzoic, anthranilic, mesylic, salicylic, p-hydroxybenzoic, phenylacetic, mandelic, ambonic, pamoic, pantothenic, sulfanilic, cyclohexylaminosulfonic, stearic, algenic, p-hydroxybutyric, galactaric, and galacturonic acids. Suitable pharmaceutically acceptable base addition salts of the compounds of the present invention include metallic salts made from lithium, sodium, potassium, magnesium, calcium, aluminium, and zinc, and organic salts made from organic bases such as choline, diethanolamine, morpholine. Alternatively, organic salts made from N,N′-dibenzylethylenediamine, chloroprocaine, choline, diethanolamine, ethylenediamine, meglumine (N-methylglucamine), procaine, ammonium salts, quaternary salts such as tetramethylammonium salt, amino acid addition salts such as salts with glycine and arginine.

For example, alkali metal salts (K, Na) and alkaline earth metal salts (Ca, Mg) may be used, but again any pharmaceutically acceptable, non-toxic salt may be used. The Na- and Ca-salts are preferred.

Applicable esters are, for example,

Straight of branched C₁-C₁₈ alkyl esters, e.g. methyl, ethyl, propyl, isopropyl, butyl, isobutyl, amyl, hexyl, heptl, octyl, nonyl, decyl, lauryl, myristyl, cetyl, stearyl, etc.

Straight chain or branched C₂-C₁₈ alkenyl esters, e.g. vinyl, allyl, undecenyl, oleyl, linolenyl, etc.

C₃-C₈ cycloalkyl esters, e.g. cyclopropyl, cyclobutyl, cyclopentyl, cyclohexyl, cycloheptyl and cyclooctyl, etc.

Aryl esters, e.g. phenyl, toluyl, xylyl, naphthyl, etc.

Alicyclic esters, e.g. menthyl, etc., or

Aralykyl esters, e.g. benzyl, phenethyl, etc.

Suitable esters may include, for example, acetates, citrates, lactates, tartrates, malonates, oxalates, salicylates, propionates, succinates, fumarates, maleates, methylene-bis-β-hydroxynaphthoates, gestisates, isethionates, di-p-toluoyltartrates, methanesulphonates, ethanesulphonates, benzenesulphonates, p-toluenesulphonates, cyclohexylsulphamates and quinates. Preferably, the ester is formed at the carboxyl group of ASA.

In a particularly preferred embodiment, the pharmaceutically acceptable salt is mesalamine hydrochloride.

An ASA derivative includes any substitution at the amine, carboxy and/or hydroxyl positions, or further substitution of the ring. For example, such substitutions may include, but are not limited to halo, alkyl, alkyoxy, alkenyl, alkynyl, cyano, hydroxyl and alkylamino. More than one substitution may occur where possible, such as on the ASA amine.

Pharmaceutically acceptable solvates, including hydrates, of such compounds and such salts are also intended to be included within the scope of this invention.

The phrase ‘therapeutically effective amount’ generally refers to an amount of a ASA, ASA derivative, or pharmaceutically acceptable salt, ester, amide, polymorph and/or prodrug thereof of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein.

Typically, a therapeutically effective dosage is formulated to contain a concentration (by weight) that allows a concentration of between about 1 mM to about 10 mM to penetrate to the basal layer of the skin.

Typically, a composition of the invention, or composition for use in a method or use of the invention, contains 1, 2, 5 or 10 mM ASA, ASA derivative, or pharmaceutically acceptable salt, ester, amide, polymorph and/or prodrug thereof (preferably the ASA is mesalamine). Preferably, the amount of ASA present in a composition, e.g. for topical application, is up to about 10% w/v, preferably up to about 10% w/v, preferably about 5% w/v, preferably about 2% w/v. Typically, the concentration (by weight) in any composition described herein is at least about 0.1% up to about 10% or more, and all combinations and subcombinations of ranges therein. The compositions can be formulated to contain ASA, ASA derivative, or pharmaceutically acceptable salt, ester, amide, polymorph and/or prodrug thereof in a concentration of from about 0.1 to less than about 20%, for example, about 19, 18, 17, 16, 15, 14, 13, 12, 11 and 10%, with concentrations of from greater than about 0.1%, for example, about 0.2, 0.3, 0.4 or 0.5%, to less than about 10%, for example, about 9, 8, 7, 6, 5, 4, 3, 2, or 1%. Exemplary compositions may contain from about 0.5% to less than about 10%, for example, about 9, 8, 7, 6, 5, 4, 3, 2, or 1%, with concentrations of from greater than about 0.5%, for example, about 0.6, 0.7, 0.8, 0.9 or 1%, to less than about 20%, for example, about 19, 18, 17, 16, 15, 14, 13, 12, 11 or 10%. The compositions can contain from greater than about 1% for example, about 2%, to less than about 10%, for example about 9 or 8%, including concentrations of greater than about 2%, for example, about 3 or 4%, to less than about 8%, for example, about 7 or 6%. The active agent can, for example, be present in a concentration of about 2% or 5%. In all cases, amounts may be adjusted to compensate for differences in amounts of active ingredients actually delivered to the treated tissue.

Frequency of application of any one of the compounds or compositions described herein, including Examples 3, 4 and 5, and includes up to about 12 times a day. The compound or composition described herein may be applied twice a day at an 8 hr interval. Typically, the composition is applied every 1, 2 or 3 hours. The frequency of application may be progressively reduced as the skin barrier improves. The frequency of application is at least maintained at a level such that at least one biochemically or clinically observable symptoms is improved compared to the start of treatment.

The words ‘treat’ or ‘treatment’ refer to therapeutic treatment wherein the object is to slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Treatment can also mean prolonging survival as compared to expected survival if not receiving treatment. Treatment may not necessarily result in the complete clearance of a disease or disorder but may reduce or minimise complications and side effects of infection and the progression of a disease or disorder. The success or otherwise of treatment may be monitored by, amongst other things, physical examination of the individual, cytopathological, serological DNA, or mRNA detection techniques. Examples of treatment of various individuals is described in the Examples, including Examples 3, 4 and 5.

Treatment of a disease or condition described herein may include a visibly, clinically or biochemically detectable change in the subject in any one or more of the following skin thickness, skin pliability, skin redness and presence of scales or scabs on the surface of the skin. Further, the treatment may also include a biochemically detectable change in the epidermis of the skin including, but not limited to, improvement in keratinocyte differentiation. Keratinocyte differentiation may be measure using any one of the markers described in the Examples such as Keratin 10. Further, a reduction in cornified layer thickness may be observed.

Improvement of the appearance of the skin includes any visibly detectable change in the colour or texture of the skin. For example, an improvement may be a visibly detectable reduction in the redness, amount or size of scales or scabs, or amount or size of cracks in the skin. As another example, the cornified envelope may shed more naturally and/or require less mechanical intervention, such as of chemical peels. Improvement may also be that the skin becomes more flexible, less itchy and/or require less frequent emollient treatment (if being used) or less frequent bathing.

Treatment of skin diseases that exhibit parakeratosis by a method, use or composition of the invention may promote orthokeratotic differentiation. Orthokeratotic differentiation may be a reduction in the cornified layer thickness, promotion of the granular layer, and/or normalisation of differentiation markers.

Treatment of lamellar ichthyosis may include reduced hyper-thickening of the cornified layer.

Preferably the subject that is treated does not have any one or more of the following: acute inflammatory large bowel disease, Crohn's Disease, Crohn's colitis, ulcerative colitis, ulcerative proctosigmoiditis, left-sided ulcerative colitis, ulcerative proctitis, psoriasis and Crohn's ileitis.

Although the invention finds application in humans, the invention is also useful for therapeutic veterinary purposes. The invention is useful for domestic or farm animals such as cattle, sheep, horses and poultry; for companion animals such as cats and dogs; and for zoo animals. Examples of breeds of dog include Golden retrievers, American bulldogs, Jack Russell terriers, and Cairn terriers.

The words ‘prevent’ and ‘prevention’ generally refer to prophylactic or preventative measures for protecting or precluding an individual not having a given disease or disorder from progressing to that disease or disorder.

The phrase ‘pharmaceutically acceptable’ indicates that the substance or composition must be compatible chemically and/or toxicologically, with the other ingredients comprising a formulation, and/or the mammal being treated therewith.

In any method or use of the invention, the administration may be in conjunction with a retinoid. Preferably, the retinoid is one that is used for treating an ichthyosis, preferably, HI. Preferably, the retinoid is acitretin, etretinate, isotretinoin or tazarotene. The retinoid may be administered systemically or topically, for example tazarotene may be administered topically. Typically, the retinoid is present in the composition at a dose lower than when it is used as a monotherapy or when used in a therapy as the only active ingredient.

Any oil or lipid emollient that is capable of creating an artificial external skin barrier when applied to the skin is contemplated is suitable for use as a compound for increasing the barrier function of the skin. Preferably, the oil or lipid emollient is applied, for example, to address the loss of water-proofing extracellular lipids in the stratum corneum. Examples of emollients suitable for use include white paraffin wax, glycerol, synthetic or plant derived ceramide/lipids, and/or emu oil.

Pharmaceutical compositions may be formulated for any appropriate route of administration including, for example, topical (for example, transdermal or ocular), oral, buccal, nasal, vaginal, rectal or parenteral administration. The term parenteral as used herein includes subcutaneous, intradermal, intravascular (for example, intravenous), intramuscular, spinal, intracranial, intrathecal, intraocular, periocular, intraorbital, intrasynovial and intraperitoneal injection, as well as any similar injection or infusion technique. In certain embodiments, compositions in a form suitable for oral use or parenteral use are preferred. Suitable oral forms include, for example, tablets, troches, lozenges, aqueous or oily suspensions, dispersible powders or granules, emulsions, hard or soft capsules, or syrups or elixirs. Within yet other embodiments, compositions provided herein may be formulated as a lyophilizate.

The various dosage units are each preferably provided as a discrete dosage tablet, capsules, lozenge, dragee, gum, or other type of solid formulation. Capsules may encapsulate a powder, liquid, or gel. The solid formulation may be swallowed, or may be of a suckable or chewable type (either frangible or gum-like). The present invention contemplates dosage unit retaining devices other than blister packs; for example, packages such as bottles, tubes, canisters, packets. The dosage units may further include conventional excipients well-known in pharmaceutical formulation practice, such as binding agents, gellants, fillers, tableting lubricants, disintegrants, surfactants, and colorants; and for suckable or chewable formulations.

Compositions intended for oral use may further comprise one or more components such as sweetening agents, flavouring agents, colouring agents and/or preserving agents in order to provide appealing and palatable preparations. Tablets contain the active ingredient in admixture with physiologically acceptable excipients that are suitable for the manufacture of tablets. Such excipients include, for example, inert diluents such as calcium carbonate, sodium carbonate, lactose, calcium phosphate or sodium phosphate, granulating and disintegrating agents such as corn starch or alginic acid, binding agents such as starch, gelatine or acacia, and lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatine capsules wherein the active ingredient is mixed with an inert solid diluent such as calcium carbonate, calcium phosphate or kaolin, or as soft gelatine capsules wherein the active ingredient is mixed with water or an oil medium such as peanut oil, liquid paraffin or olive oil.

Aqueous suspensions contain the active ingredient(s) in admixture with excipients suitable for the manufacture of aqueous suspensions. Such excipients include suspending agents such as sodium carboxymethylcellulose, methylcellulose, hydropropylmethylcellulose, sodium alginate, polyvinylpyrrolidone, gum tragacanth and gum acacia, and dispersing or wetting agents such as naturally-occurring phosphatides (for example, lecithin), condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol mono-oleate, or condensation products of ethylene oxide with partial esters derived from fatty acids and hexitol anhydrides such as polyethylene sorbitan monooleate. Aqueous suspensions may also comprise one or more preservatives, for example ethyl, or n-propyl p-hydroxybenzoate, one or more colouring agents, one or more flavouring agents, and one or more sweetening agents, such as sucrose or saccharin.

Oily suspensions may be formulated by suspending the active ingredients in a vegetable oil such as arachis oil, olive oil, sesame oil or coconut oil, or in a mineral oil such as liquid paraffin. The oily suspensions may contain a thickening agent such as beeswax, hard paraffin or cetyl alcohol. Sweetening agents such as those set forth above, and/or flavouring agents may be added to provide palatable oral preparations. Such suspensions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavouring and colouring agents, may also be present.

Pharmaceutical compositions may also be in the form of oil-in-water emulsions. The oily phase may be a vegetable oil such as olive oil or arachis oil, a mineral oil such as liquid paraffin, or a mixture thereof. Suitable emulsifying agents include naturally-occurring gums such as gum acacia or gum tragacanth, naturally-occurring phosphatides such as soy bean lecithin, and esters or partial esters derived from fatty acids and hexitol, anhydrides such as sorbitan monoleate, and condensation products of partial esters derived from fatty acids and hexitol with ethylene oxide such as polyoxyethylene sorbitan monoleate. An emulsion may also comprise one or more sweetening and/or flavouring agents.

Syrups and elixirs may be formulated with sweetening agents, such as glycerol, propylene glycol, sorbitol or sucrose. Such formulations may also comprise one or more demulcents, preservatives, flavouring agents and/or colouring agents.

Compounds may be formulated for local or topical administration, such as for topical application to the skin. Formulations for topical administration typically comprise a topical vehicle combined with active agent(s), with or without additional optional components. For topical application, preferably ASA, ASA derivative, or a pharmaceutically acceptable salt, ester, amide or polymorph thereof is used. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof. For local (i.e. not systemic) action such as a topical route of application, prodrugs that are capable of being broken down by enzymes present in the skin, preferably the epidermis or dermis, into the active component can be used. Alternatively, prodrugs capable of being broken down by another component in the formation during drug delivery may be used. Appropriate prodrugs may include sulfasalazine, balsalazide, olsalazine if appropriate for the desired route of administration. Other prodrugs prepared through common variations to the ASA structure will be well-known to a person skilled in the art and are included herein. For example, the types of prodrugs described in Zawilska, J. B. et al. Pharmacological Reports, 2013, 65, 1-14 are encompassed in this application where they are relevant to the ASA structure and route of administration.

Suitable topical vehicles and additional components are well known in the art, and it will be apparent that the choice of a vehicle will depend on the particular physical form and mode of delivery. Topical vehicles include organic solvents such as alcohols (for example, ethanol, iso-propyl alcohol or glycerine), glycols such as butylene, isoprene or propylene glycol, aliphatic alcohols such as lanolin, mixtures of water and organic solvents and mixtures of organic solvents such as alcohol and glycerine, lipid-based materials such as fatty acids, acylglycerols including oils such as mineral oil, and fats of natural or synthetic origin, phosphoglycerides, sphingolipids and waxes, protein-based materials such as collagen and gelatine, silicone-based materials (both nonvolatile and volatile), and hydrocarbon-based materials such as microsponges and polymer matrices.

A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatine-microcapsules, liposomes, albumin microspheres, microemulsions, nanoparticles or nanocapsules.

A topical formulation may be prepared in a variety of physical forms including, for example, solids, pastes, creams, foams, lotions, gels, powders, aqueous liquids, emulsions, sprays and skin patches. The physical appearance and viscosity of such forms can be governed by the presence and amount of emulsifier(s) and viscosity adjuster(s) present in the formulation. Solids are generally firm and non-pourable and commonly are formulated as bars or sticks, or in particulate form. Solids can be opaque or transparent, and optionally can contain solvents, emulsifiers, moisturizers, emollients, fragrances, dyes/colorants, preservatives and other active ingredients that increase or enhance the efficacy of the final product. Creams and lotions are often similar to one another, differing mainly in their viscosity. Both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams, may also contain solvents, emulsifiers, moisturizers, emollients, fragrances, dyes/colorants, preservatives and other active ingredients that increase or enhance the efficacy of the final product. Liquids are thinner than creams, lotions, or gels, and often do not contain emulsifiers. Liquid topical products often contain solvents, emulsifiers, moisturizers, emollients, fragrances, dyes/colorants, preservatives and other active ingredients that increase or enhance the efficacy of the final product.

Emulsifiers for use in topical formulations include, but are not limited to, ionic emulsifiers, cetearyl alcohol, non-ionic emulsifiers like polyoxyethylene oleyl ether, PEG-40 stearate, ceteareth-12, ceteareth-20, ceteareth-30, ceteareth alcohol, PEG-100 stearate and glyceryl stearate. Suitable viscosity adjusting agents include, but are not limited to, protective colloids or nonionic gums such as hydroxyethylcellulose, xanthan gum, magnesium aluminum silicate, silica, microcrystalline wax, beeswax, paraffin, and cetyl palm itate. A gel composition may be formed by the addition of a gelling agent such as chitosan, methyl cellulose, ethyl cellulose, polyvinyl alcohol, polyquaterniums, hydroxyethylceilulose, hydroxypropylcellulose, hydroxypropylmethylcellulose, carbomer or ammoniated glycyrrhizinate. Suitable surfactants include, but are not limited to, nonionic, amphoteric, ionic and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate may be used within topical formulations.

Preservatives include, but are not limited to, antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. Suitable moisturizers include, but are not limited to, lactic acid and other hydroxy acids and their salts, glycerine, propylene glycol, and butylene glycol. Suitable emollients include lanolin alcohol, lanolin, lanolin derivatives, cholesterol, petrolatum, isostearyl neopentanoate and mineral oils. Suitable fragrances and colours include, but are not limited to, FD&C Red No. 40 and FD&C Yellow No. 5. Other suitable additional ingredients that may be included in a topical formulation include, but are not limited to, abrasives, absorbents, anticaking agents, antifoaming agents, antistatic agents, astringents (such as witch hazel), alcohol and herbal extracts such as chamomile extract, binders/excipients, buffering agents, chelating agents, film forming agents, conditioning agents, propellants, opacifying agents, pH adjusters and protectants.

Typical modes of delivery for topical compositions include application using the fingers, application using a physical applicator such as a cloth, tissue, swab, stick or brush, spraying including mist, aerosol or foam spraying, dropper application, sprinkling, soaking, and rinsing. Controlled release vehicles can also be used, and compositions may be formulated for transdermal administration (for example, as a transdermal patch).

A pharmaceutical composition may be formulated as inhaled formulations, including sprays, mists, or aerosols. For inhalation formulations, the composition or combination provided herein may be delivered via any inhalation methods known to a person skilled in the art. Such inhalation methods and devices include, but are not limited to, metered dose inhalers with propellants such as CFC or HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and may be filled into conventional aerosol containers that are closed by a suitable metering valve.

An example of a composition for topical use in a method or use described herein includes a skin moisturising cream as a carrier for ASA, ASA derivative, or pharmaceutically acceptable salt, ester, amide, polymorph and/or prodrug thereof. Preferably, the cream comprises 25% stearyl alcohol, 25% petroleum jelly, 12% glycerin, 5% Tween 80, and 33% distilled water, and ASA, ASA derivative, or pharmaceutically acceptable salt, ester, amide, polymorph and/or prodrug thereof. This cream formulation is akin to that normally used by HI patients as part of their normal disease management regime.

A further example of a topical cream including Emu Oil (preferably at 25%), Stearyl Alcohol (preferably at 22.5%), petroleum jelly (preferably at 12.5%), glycerine (preferably at 15%), Tween 80 (preferably at 5%), water (preferably at 20%) and ASA, ASA derivative, or pharmaceutically acceptable salt, ester, amide, polymorph and/or prodrug thereof preferably at 1, 2, 3, 4 or 5%. Preferably the ASA is mesalamine.

Inhalant compositions may comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent such as isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable formulations, wherein the carrier is a liquid, for administration, as for example, a nasal spray or as nasal drops, include aqueous or oily solutions of the active ingredient.

Pharmaceutical compositions may also be prepared in the form of suppositories such as for rectal administration. Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

Pharmaceutical compositions may be formulated as sustained release formulations such as a capsule that creates a slow release of modulator following administration. Such formulations may generally be prepared using well-known technology and administered by, for example, oral, rectal or subcutaneous implantation, or by implantation at the desired target site. Carriers for use within such formulations are biocompatible, and may also be biodegradable. Preferably, the formulation provides a relatively constant level of modulator release. The amount of modulator contained within a sustained release formulation depends upon, for example, the site of implantation, the rate and expected duration of release and the nature of the condition to be treated or prevented.

In another embodiment there is provided a kit or article of manufacture including ASA, ASA derivative, or pharmaceutically acceptable salt, ester, amide, polymorph and/or prodrug thereof and/or pharmaceutical composition as described above. Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

In other embodiments there is provided a kit for use in a therapeutic application mentioned above, the kit including:
a container holding a therapeutic composition in the form of ASA, ASA derivative, or pharmaceutically acceptable salt, ester, amide, polymorph and/or prodrug thereof or pharmaceutical composition;
a label or package insert with instructions for use.

Preferably, the ASA is mesalamine, 4-ASA or 3-ASA, derivative, or pharmaceutically acceptable salt, ester, amide, polymorph and/or prodrug thereof. More preferably, the ASA is mesalamine, mesalamine derivative, or pharmaceutically acceptable salt, ester, amide, polymorph and/or prodrug thereof.

In certain embodiments the kit may contain one or more further active principles or ingredients for treatment of the skin condition.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds a therapeutic composition which is effective for treating the condition and may have a sterile access port (for example the container may be an intravenous solution bag or a vial having a stopper pierceable by a hypodermic injection needle). The label or package insert indicates that the therapeutic composition is used for treating the condition of choice. In one embodiment, the label or package insert includes instructions for use and indicates that the therapeutic composition can be used to treat a skin condition described herein.

The kit may comprise (a) a therapeutic composition; and (b) a second container with a second active principle or ingredient contained therein. The kit in this embodiment of the invention may further comprise a package insert indicating that the and other active principle can be used to treat a disorder or prevent a complication stemming from a skin condition described herein. Alternatively, or additionally, the kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain embodiments the therapeutic composition may be provided in the form of a device, disposable or reusable, including a receptacle for holding the therapeutic or pharmaceutical composition. In one embodiment, the device is a syringe. The device may hold 1-2 mL of the therapeutic composition. The therapeutic composition may be provided in the device in a state that is ready for use or in a state requiring mixing or addition of further components.

It will be understood, that the specific dose level for any particular patient will depend upon a variety of factors including the activity of the specific compound employed, the age, body weight, general health, sex, diet, time of administration, route of administration, and rate of excretion, drug combination (i.e. other drugs being used to treat the patient), and the severity of the particular disorder undergoing therapy.

It will be understood that the following examples are intended to demonstrate these and other aspects of the invention and although the examples describe certain embodiments of the invention, it will be understood that the examples do not limit these embodiments to these things. Various changes can be made and equivalents can be substituted and modifications made without departing from the aspects and/or principles of the invention mentioned above. All such changes, equivalents and modifications are intended to be within the scope of the claims set forth herein.

### Example 1

Mouse Strains

The mouse strain ‘Abca12^{tm1Lex}’ NIH-0129 was obtained from Lexicon genetics and are referred to herein as Abca12Lx12/Lx12, or simply Lx12/Lx12, mice. These mice have a puromycin selection cassette-mediated exon 8 disruption and recapitulate the features of HI akin to other Abca12 mutant strains. All animal procedures complied with standards set under Australian guidelines for animal welfare and experiments were subject to Monash University animal welfare ethics review panels.

Antibodies and Stains

Anti-cleaved caspase 3 (#9664P) 1:100 (IHC) Cell Signalling Technologies, USA. Anti-filaggrin (PRB-417P) 1: 1000 (IHC) Covance, USA. Anti-involucrin (PRB-140C) 1: 1000 (IHC) Covance, USA. Antikeratin 10 (PRB-159P) 1: 500 (IHC) Covance, USA. Antikeratin 10 (sc-23877) 1: 100 (IHC) Santa Cruz Biotechnology USA. Anti-keratin 14 (LL002) (ab7800) 1: 250-1000 (IHC) Abcam, UK. Anti-loricrin (PRB-145P) 1: 1000 (IHC) Covance, USA. Molecular Probes AlexaFluor A488 and 555 secondary antibodies raised in Donkey, against rabbit or mouse, were used at 1: 600, from Life Technologies. Nuclei stains used included DAPI (Sigma-Aldrich) 1: 1000. DAB staining was performed by the Monash Histology Platform using Leica autostainers and Dako products.

### Example 2

The mouse models used in the experiments described below include Lx12/Lx12 which models HI and Lx12/+ which represents seemingly normal mice carrying the recessive HI mutant allele. The Lx12/+ shows subtle sub-pathogenic alterations in keratinocyte differentiation and lipid dysfunction. This is a model of a much less severe form of HI. The results with both of these models show that the treatment with mesalamine is beneficial in models of varying disease severity and that mesalamine treatment can completely or partially correct skin differentiation defects.

The ability of mesalamine to rescue the impaired differentiation in an HI mouse model, which represents an extremely severe inflammatory skin disease, was tested. Embryonic back skins at E16.5 were harvested from a litter containing wild type, heterozygous and HI embryos. The skin was divided into two pieces and each cultured for 4 days in a standard ex vivo chamber assay, where the skin rests dermis-side down on a chamber insert membrane and draws cell culture media up from the below well, forming an air face on the epidermis side and liquid face for the dermal side, similar to the normal skin environment. Mesalamine was added in the media of one of each pair of skins at 10 mM (the active concentration determined to have most impact in the gut for intestinal diseases). When comparing the effects of mesalamine on each pair matched skin and across genotypes, it was clear that mesalamine significantly rescued skin differentiation defects in HI.

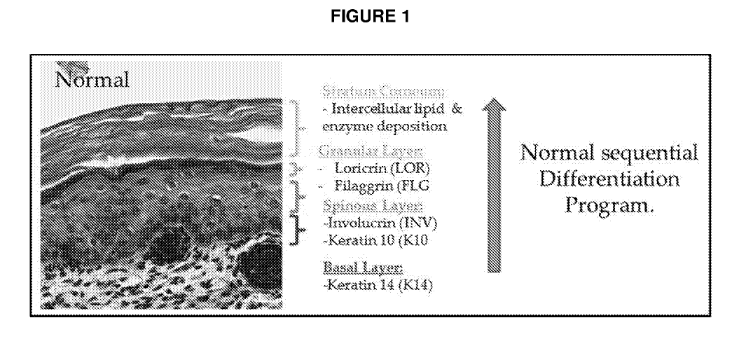

FIG. 1 shows an overview of normal keratinocyte differentiation. The epidermis portion of skin is composed of 4 distinct layers and keratinocytes progressively move upwards through these phases starting at the basal layer (where proliferation normally occurs) until being shed as a dead husk at the top of the stratum corneum. Each phase has a particular biochemical identity.

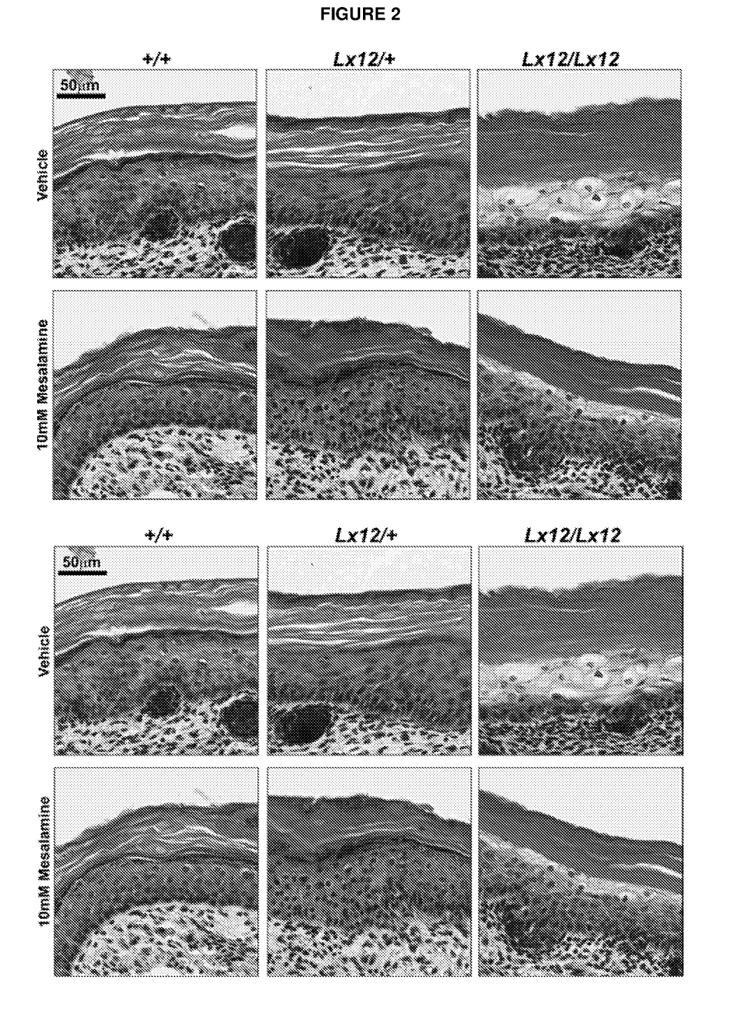

Haematoxylin and Eosin staining of E16.5 embryonic mouse dorsal skin cultured ex vivo for 4 days on chamber inserts, with 10 mM mesalamine in standard culture media or vehicle alone. +/+ presents wild type skin. Lx12/Lx12 represents the HI mutant skin and Lx12/+ represents seemingly normal skin carrying the recessive HI mutant allele (FIG. 2). Note the abnormality observed in the spinous layer, loss of the granular layer and thickening of the stratum corneum in Lx12/Lx12 (HI) vehicle treated skin. However upon mesalamine treatment the HI epidermis acquires a more-normal appearance. Mesalamine did not however appear to greatly effect+/+ skin. Images are repeated in grayscale.

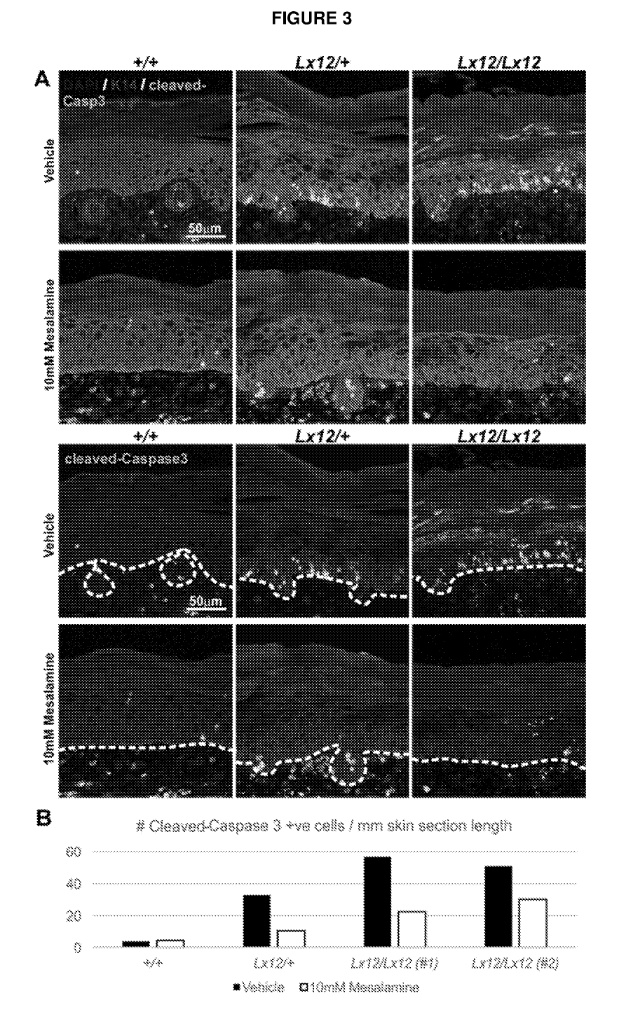

Immunofluorescent staining of E16.5 embryonic mouse dorsal skin cultured ex vivo for 4 days on chamber inserts, with 10 mM mesalamine in standard culture media or vehicle alone. +/+ presents wild type skin. Lx12/Lx12 represents the HI mutant skin and Lx12/+ represents seemingly normal skin carrying the recessive HI mutant allele. FIG. 3 (A) in Red the Keratin 14 (K14) basal epidermal marker is observed, in Blue is the nuclear dye, DAPI and in Green is the marker of apoptosis (programed cell death), cleaved-Caspase-3 (cleaved-Casp3). The images are also repeated with focus only on cleaved-Caspase-3 in greyscale. Dashed line indicates boundary between epidermis and dermis. FIG. 3 (B) shows the quantification of the number of apoptotic (cleaved-Caspase-3 positive) cells observed, across the skin section length for four siblings of +/+, Lx12/+ and Lx12/Lx12 (x2) genotypes. Note: The epidermis is normally very resistant to apoptosis, however there are an increasing number of apoptotic keratinocytes observed in Lx12/+ and Lx12/Lx12 (HI) vehicle treated skin. Upon mesalamine treatment however, apoptosis is reduced.

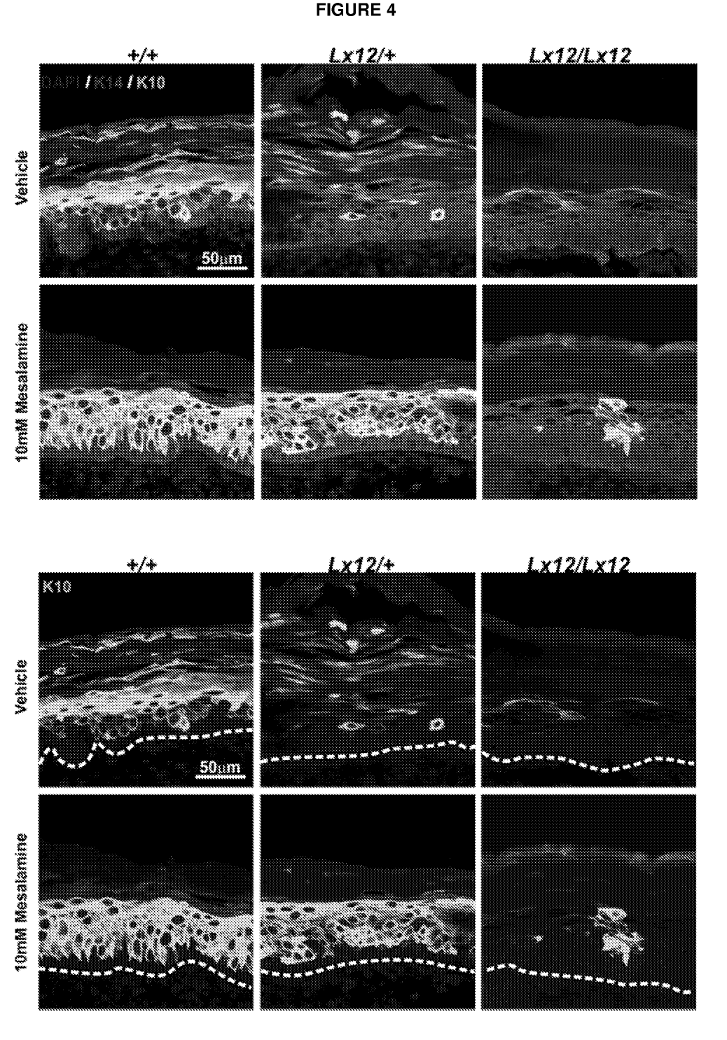

As shown in FIG. 4, immunofluorescent staining of E16.5 embryonic mouse dorsal skin cultured ex vivo for 4 days on chamber inserts, with 10 mM mesalamine in standard culture media or vehicle alone. +/+ presents wild type skin. Lx12/Lx12 represents the HI mutant skin and Lx12/+ represents seemingly normal skin carrying the recessive HI mutant allele. In Red the Keratin 14 (K14) basal epidermal marker is observed, in Blue is the nuclear dye, DAPI and in Green is the spinous layer marker, Keratin 10 (K10). The images are also repeated with focus only on K10 in greyscale. Dashed line indicates boundary between epidermis and dermis. Note the decreasing number of K10+ve keratinocytes observed in both Lx12/+ and Lx12/Lx12 (HI) vehicle treated skin. Upon mesalamine treatment however, K10 cells revert to normal levels in Lx12/+ skin, and some modest improvement is seen in Lx12/Lx12 skin.

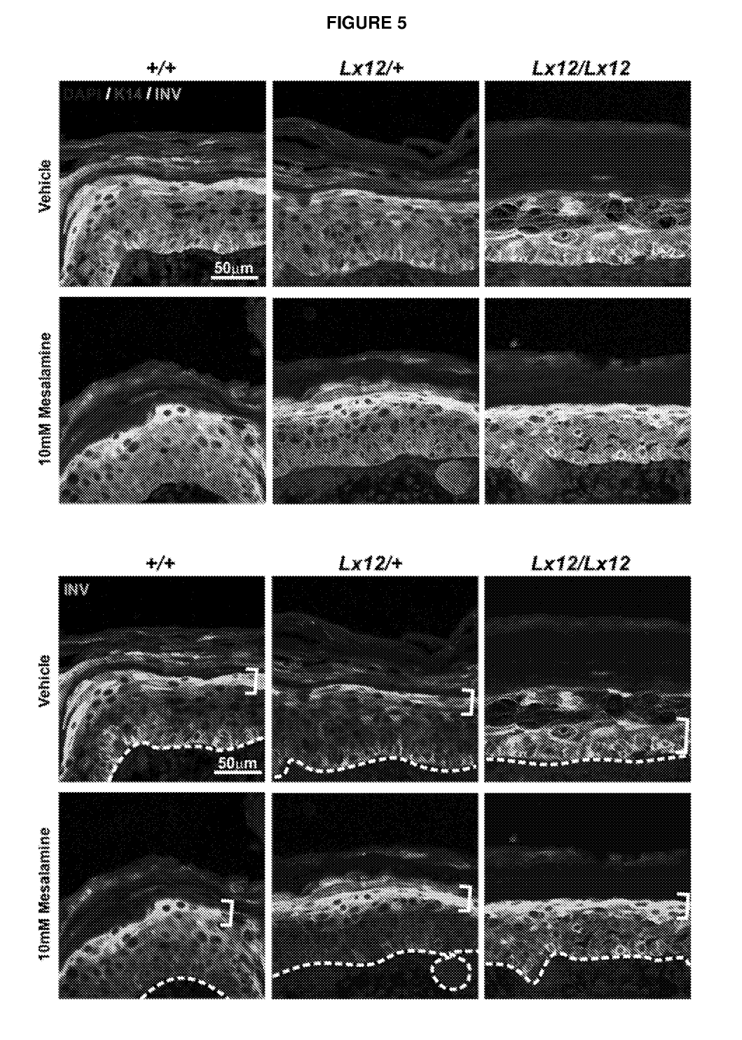

FIG. 5 shows immunofluorescent staining of E16.5 embryonic mouse dorsal skin cultured ex vivo for 4 days on chamber inserts, with 10 mM mesalamine in standard culture media or vehicle alone. +/+ presents wild type skin. Lx12/Lx12 represents the HI mutant skin and Lx12/+ represents seemingly normal skin carrying the recessive HI mutant allele. In Red the Keratin 14 (K14) epidermal marker is observed, in Blue is the nuclear dye, DAPI and in Green is the spinous and granular layer marker, Involucrin (INV). The images are also repeated with focus only on INV in greyscale. Dashed line indicates boundary between epidermis and dermis. Brackets indicate predominant expression of INV. Note: INV is most strongly detected in the granular layers but exhibits disorganised and premature expression in Lx12/Lx12 (HI) vehicle treated skin. Upon mesalamine treatment however, a more organised, and delayed (granular layer) expression of INV is seen in Lx12/Lx12 skin.

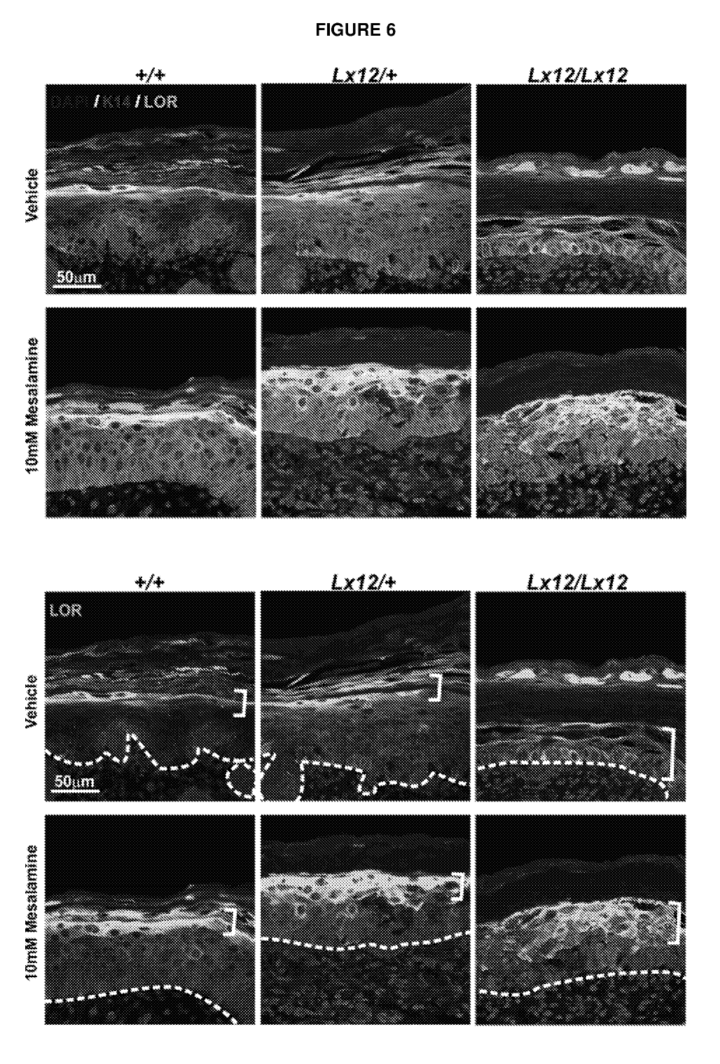

Immunofluorescent staining of E16.5 embryonic mouse dorsal skin cultured ex vivo for 4 days on chamber inserts, with 10 mM mesalamine in standard culture media or vehicle alone. +/+ presents wild type skin. Lx12/Lx12 represents the HI mutant skin and Lx12/+ represents seemingly normal skin carrying the recessive HI mutant allele. In Red the Keratin 14 (K14) epidermal marker is observed, in Blue is the nuclear dye, DAPI and in Green is the granular layer marker, Loricrin (LOR) (FIG. 6). The images are also repeated with focus only on LOR in greyscale. Dashed line indicates boundary between epidermis and dermis. Brackets indicate predominant expression of LOR. Note: LOR is most strongly detected in the granular layers and to lesser extent in the stratum corneum. Premature expression of LOR in basal and suprabasal keratinocytes of Lx12/Lx12 (HI) vehicle treated skin is observed, however, upon mesalamine treatment a more normal, predominantly granular layer expression of LOR is seen in Lx12/Lx12 skin.

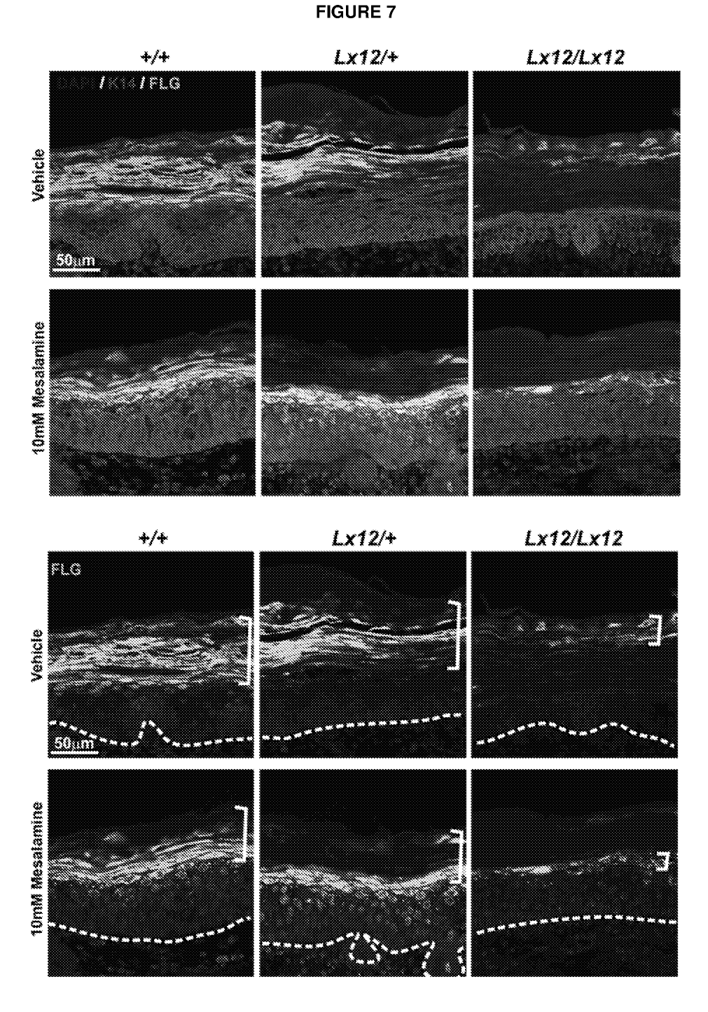

FIG. 7 shows immunofluorescent staining of E16.5 embryonic mouse dorsal skin cultured ex vivo for 4 days on chamber inserts, with 10 mM mesalamine in standard culture media or vehicle alone. +/+ presents wild type skin. Lx12/Lx12 represents the HI mutant skin and Lx12/+ represents seemingly normal skin carrying the recessive HI mutant allele. In Red the Keratin 14 (K14) epidermal marker is observed, in Blue is the nuclear dye, DAPI and in Green is the granular to cornified layer marker, Filaggrin (FLG). The images are also repeated with focus only on FLG in greyscale. Dashed line indicates boundary between epidermis and dermis. Brackets indicate predominant expression of FLG. Note: Filaggrin is most strongly detected in the granular layers and to lesser extent in the stratum corneum. Progressive loss of granular layer FLG in both Lx12/+ and Lx12/Lx12 (HI) vehicle treated skin, is observed, however, upon mesalamine treatment granular layer FLG is restored to Lx12/+ skin and partially restored to Lx12/Lx12 skin.

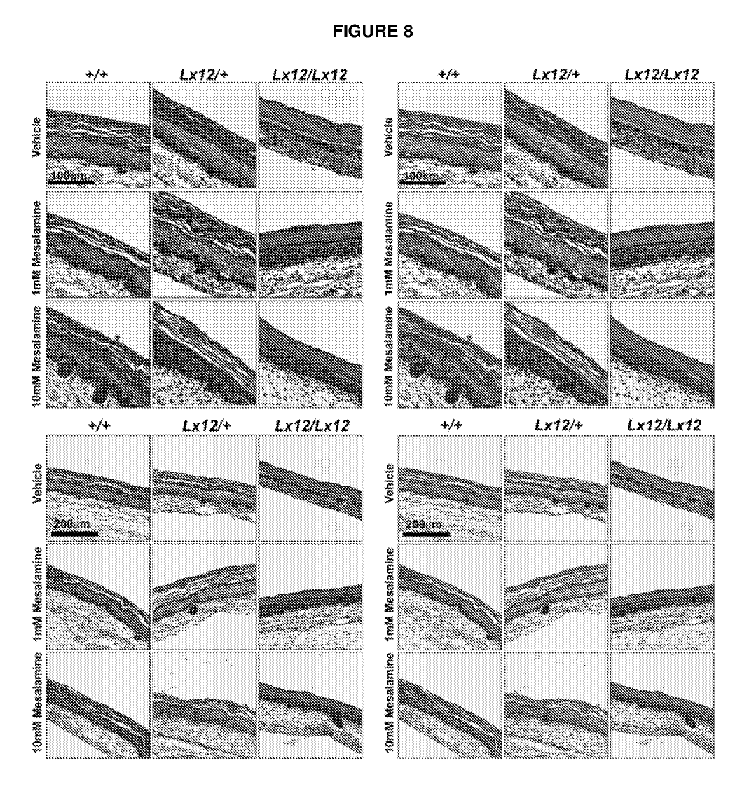

Haematoxylin and Eosin staining of embryonic mouse dorsal skin from a second E16.5 litter, cultured ex vivo for 4 days on chamber inserts, with 1 or 10 mM mesalamine in standard culture media or vehicle alone. +/+ presents wild type skin is shown in FIG. 8. Lx12/Lx12 represents the HI mutant skin and Lx12/+ represents seemingly normal skin carrying the recessive HI mutant allele. Note the abnormality observed in the spinous layer, loss of the granular layer and proportional thickening of the stratum corneum in Lx12/Lx12 (HI) vehicle treated skin. However upon mesalamine treatment the epidermis acquires a more-normal appearance, with the dose of 1 mM and 10 mM being effective. Representative images are provided at two magnifications to highlight the uniformity of the effects across the skin, and repeated in grayscale.

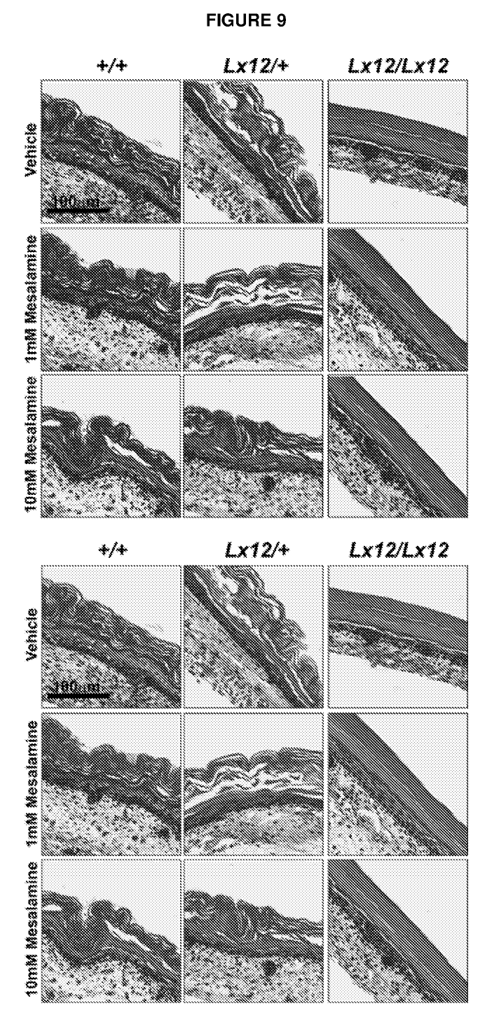

Representative Haematoxylin and Eosin staining of embryonic mouse dorsal skin from two E18.5 litters, cultured ex vivo for 4 days on chamber inserts, with 1 or 10 mM mesalamine in standard culture media or vehicle alone is shown in FIG. 9. +/+ presents wild type skin. Lx12/Lx12 represents the HI mutant skin and Lx12/+ represents seemingly normal skin carrying the recessive HI mutant allele. Note once again the abnormality observed in the spinous layer, loss of the granular layer and proportional thickening of the stratum corneum in Lx12/Lx12 (HI) vehicle treated skins. Lx12/+ skins also appeared slightly abnormal relative to +/+ vehicle treated skins. However upon 1 mM mesalamine treatment, the HI epidermis acquires a more-normal appearance again, while 10 mM mesalamine in this context appears to affect all genotypes by reducing the keratinocytes accumulating in the spinous layers (indicative of enhanced terminal differentiation). This suggests the optimal dose will be lower than 10 mM, and likely close to 1 mM. Images are repeated in grayscale.

### Example 3

HI patient identified and prescribed mesalamine emollient cream to use in place of current moisturisers. Patient applies cream every few hours initially in same manner as previous moisturisers. Over two to three weeks, skin will naturally turnover and acquire a more normal appearance and feel. Skin redness and itching may reduce. As skin differentiation becomes more-normal, inflammation is suppressed and barrier function improved, the frequency of mesalamine emollient cream application, mechanical scale removal and hydrating baths may then be reduced to a maintenance level.

### Example 4

Pregnant mother identified with HI fetus and mesalamine injected in utero (late in pregnancy) to promote more-normal skin development prior to delivery. At delivery, in conjunction with topical emollient creams and humidicribs, fetal HI skin adaption to a terrestrial environment should be accelerated from exposure to mesalamine, inflammation should be reduced and neonate survival rates increased well above 50%. Mesalamine emollient creams can then be used in an ongoing manner as for Example 3.

### Example 5

Newborn identified with Harlequin Ichthyosis. mesalamine emollient cream in conjunction with humidicribs and mechanical scale removal. HI skin adaption to a terrestrial environment should be accelerated from exposure to mesalamine, inflammation should be reduced and neonate survival rates increased well above 50%. Mesalamine emollient creams can then be used in an ongoing manner as for Example 3.

### Example 6

Embryonic back skin was collected at E16.5 from litters generated by Abca12 lx12/+ cross Abca12 lx12/+ matings to generate Harlequin Ichthyosis (HI) embryos (Abca12 lx12/lx12) and wild type siblings (Abca12+/+). The embryonic skin was then cultured 4 days on a porous membrane chamber insert with the epidermis exposed to air and dermis side in contact with media drawn through the porous membrane to permit maturation and adaptation to air. The matured embryonic skin was then enzymatically digested to separate the epidermis and dermis. The epidermal peel was then stored frozen at −80 degrees celsius and RNA later extracted using a Trizol method. RNA sequencing was then performed on the RNA collected from 4 HI and 4 wild type epidermal peels, with the ribo-depletion method of RNA library preparation and run on an Illumina NextSeq 500 platform with 75 bp paired read format. The raw data was analysed using commercial RNAseq software to generate gene counts and perform statistical analysis. Significantly altered genes were those that where more than 1.5 fold up or down-regulated with a ttest p-value of <0.05 when comparing the two groups of HI to wild type epidermis.

TABLE 1
Genes found significantly altered in embryonic murine HI epidermis compared to wild type epidermis
*(− down regulated, + up-regulated, Harlequin Ichthyosis epidermis compared Wild type).

| | *Fold Change | p value | Mutated in Disease |
|---|---|---|---|
| Skin Disease Genes | | | |
| Abca12 | −Infinity | 0.000039 | Harlequin Ichthyosis and Lamellar Ichthyosis type 2 |
| Abhd5 (CGI-58) | −2.68 × 10⁰⁵ | 0.036 | Chanarin-Dorfman syndrome |
| Alox12b | −Infinity | 0.032 | Autosomal Recessive Congenital Ichthyosis 2 (ARCI2) |
| Aloxe3 | −3.03 × 10¹¹⁹ | 0.012 | Autosomal Recessive Congenital Ichthyosis 3 (ARCI3) |
| Flg | −Infinity | 0.000 | Ichthyosis vulgaris and dermatitis |
| Lbr | −3.61 × 10²⁶ | 0.001 | Ichthyosis vulgaris in mouse knockout model |
| Lipn | −1.58 × 10¹⁴ | 0.023 | Autosomal Recessive Congenital Ichthyosis 8 (ARCI8) |
| Nfe2l2 (Nrf2) | +1.59 × 10¹¹⁵ | 0.024 | Ichthyosis in mouse overexpression model |
| Slc27a4 (FATP4) | −7.34 × 10¹⁹⁴ | 0.039 | Ichthyosis Prematurity Syndrome |
| Smpd1 (aSMase) | −6.21 × 10⁸⁴ | 0.004 | Niemann-Pick disease |
| Tgm1 | −Infinity | 0.004 | Lamellar Ichthyosis |
| Additional Genes | | | |
| Tgm3 | −Infinity | 0.004 | related to Tgm1 (Lamellar Ichthyosis) |
| | | | abnormal hairs in mutant mouse models |
| Alox8 | −2.73 × 10³⁶ | 0.033 | related to Alox12b/Aloxe3 (ARCI 2 and 3) |
| Elovl6 | −3.36 × 10⁴⁸ | 0.013 | related to Elovl1 (Mouse knockout model of Ichthyosis) |
| Nipal2 | — | 0.036 | related to Nipal4 (ARCI6) |
| | +410557866 | | |
| | 0 | | |
| Nipal1 | −3.34 × 10¹³⁰ | 0.015 | related to Nipal4 (ARCI6) |

As expected the knockout (infinite down-regulation, -infinity) of Abca12 in Abca12 lx12/lx12 mice was detected in the RNAseq analysis confirming the validity of the system. Of further interest many additional genes, whose mutation or over-expression have been directly associated with human and/or mouse diseases that feature skin barrier defects where also altered, as well as genes in gene families associated with skin disease. As these other genes are deregulated down-stream of Abca12 mutations, this data suggests that a treatment that is corrective for Harlequin Ichthyosis may also be suitable as a skin therapy for the additional human diseases outlined in the above table and described elsewhere herein.

### Example 7

Mesalamine is able to correct Cornified Layer thickening in Grainyhead-like 3 (GRHL3) −/− epidermis (a proxy model of Lamellar Ichthyosis).

Embryonic back skin was collected at E16.5 from litters generated by GRHL3+/− cross Abca12 GRHL3+/− matings to generate Ichthyosis embryos GRHL3 −/− and control siblings GRHL3+/− and GRHL3+/+. The embryonic skin was then cultured 4 days on a porous membrane chamber insert with the epidermis exposed to air and dermis side in contact with media drawn through the porous membrane to permit maturation and adaptation to air. Skins were then harvested and fixed in 4% PFA for 3-4 hrs then stored in 80% Ethanol until processed and embedded in paraffin wax. 8 micron sections were then cut and stained with Haematoxylin and Eosin and analysed.

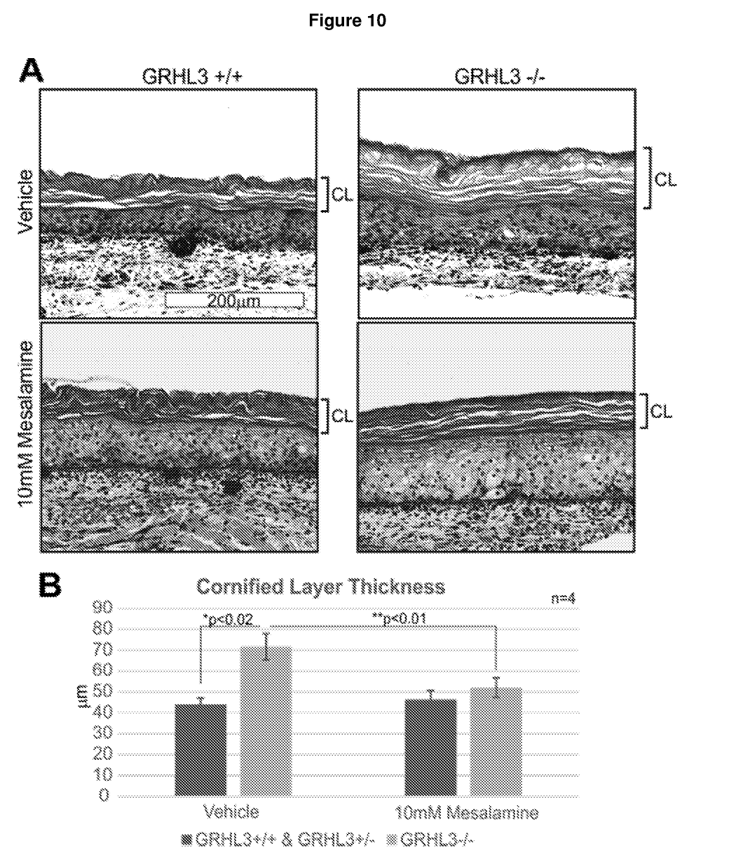

GRHL3 knockout mice also exhibit a neonatal lethal barrier defect and the defect is in part through reduced TGM1 expression, making it a proxy model of Lamellar Ichthyosis (Ting et al. Organogenesis. 2005 April; 2(2):33-5). Mesalamine treatment of this Ichthyosis mouse model also promoted signs of disease correction and reduced hyper-thickening of the cornified layer (FIG. 10).

### Example 8

Creation of Conditional Inducible Adult Mouse Model of Harlequin Ichthyosis.

The inventors developed a novel mouse model which allows us to selectively delete the Abca12 gene in adult mouse skin. Abca12^{tm1a(EUCOMM)Hmgu} modified mouse embryonic stem cells were purchased and Abca12^{tm1a(EUCOMM)Hmgu} animals subsequently derived. These mice carried an frt flanked LacZ genetrap disruption of the Abca12 gene, which was excised by crossing to Flippase mice to produce a floxed conditional allele of Abca12 (loxp sites flank exon 4), termed tm1c. Abca12 tm1c alleles are functionally wild type until exon 4 is deleted by the action of Cre Recombinase to produce a null allele termed tm1d. To generate an inducible adult skin-specific model of Harlequin Ichthyosis, the inventors have crossed our Abca12 tm1c mice with a widely available epidermis-specific Keratin 14 promoter driven Cre recombinase mouse strain where Cre function is regulated by application of tamoxifen (4OHT) through Cre-fusion with a mutant estrogen receptor ligand binding domain (termed K14-CreER). Mice of either gender aged 7-9 weeks old and in the telogen phase of the hair cycle had a small area of lower dorsal skin clipped and treated by topical application of 1.5 mg 4-hydroxy-tamoxifen (4OHT) in 100 ul of acetone or acetone vehicle alone. 4OHT was applied every second day for a total of 3 applications. Mice where then sacrificed on the 11th day of the experiment for analysis.

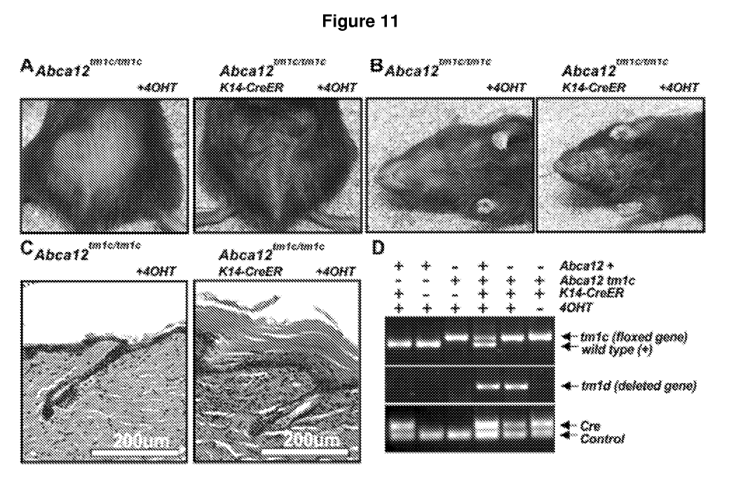

As expected only mice that inherit two Abca12 tm1c alleles, the K14-CreER transgene and are exposed to 4OHT to activate the Cre-mediated deletion of the Abca12 gene, acquire a Harlequin Ichthyosis phenotype (FIG. 11). In the absence of 4OHT and/or absence of Cre and/or presence of at least 1 Abca12+(wild type) allele, the skin remains functionally normal/wild type (FIG. 11).

### Example 9

Topical Application of Mesalamine Promotes Orthokeratosis in Mouse Tail Scale Assay.

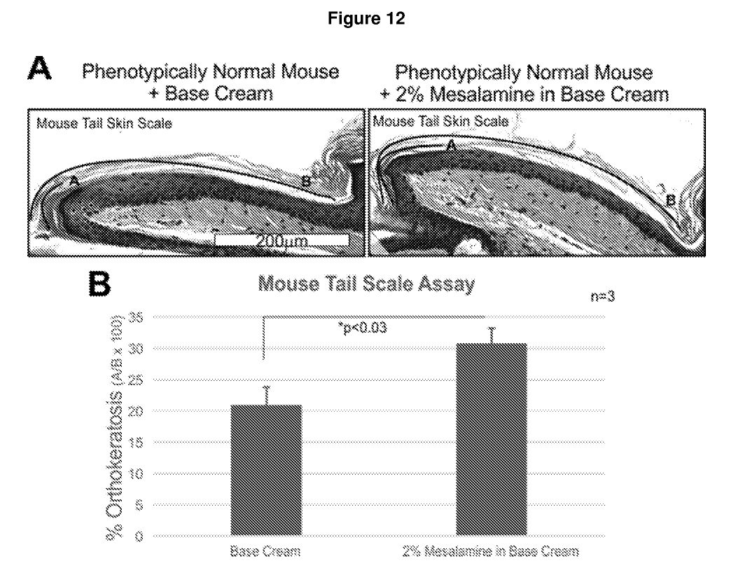

7-9 week old phenotypically normal Abca12 tm1c/tm1c mice of either gender had 100 μl of 2% Mesalamine Cream (or Base cream alone of formula 25% Emu Oil, 22.5% Stearyl Alcohol, 12.5% petroleum jelly, 15% glycerine, 5% Tween 80, 20% water) applied twice daily at an 8 hr interval for 6 days to the naturally parakeratotic tail scale epidermis. Mice were sacrificed and the tail skin tissue harvested and fixed in 4% PFA in PBS overnight, then stored in 80% Ethanol until processed and wax embedded for paraffin sectioning. 8 μm sections were cut and stained with Haematoxylin and Eosin to show tissue morphology. Images were taken from at least 10 scales from 3 mice per treatment condition and the % orthokeratosis measured as defined in the figure legend for FIG. 12.

The mouse tail scale is naturally parakeratotic (ie lacks a granular layer) and has been frequently used as a model system to test drugs for the treatment of psoriasis and other parakeratotic skin diseases, by examining the reacquisition of the granular layer as a measure of orthokeratosis. In this particular assay the treatment with a 2% Mesalamine cream twice daily for just 6 days significantly increased the proportion of scale exhibiting a granular layer from 20% to 30% (FIG. 12). This finding suggests Mesalamine can promote orthokeratotic differentiation in additional skin diseases that exhibit parakeratosis.

### Example 10

Additive Action of Mesalamine and Acitretin Increases Orthokeratosis in Ex Vivo Mouse Tail Scale Assay.

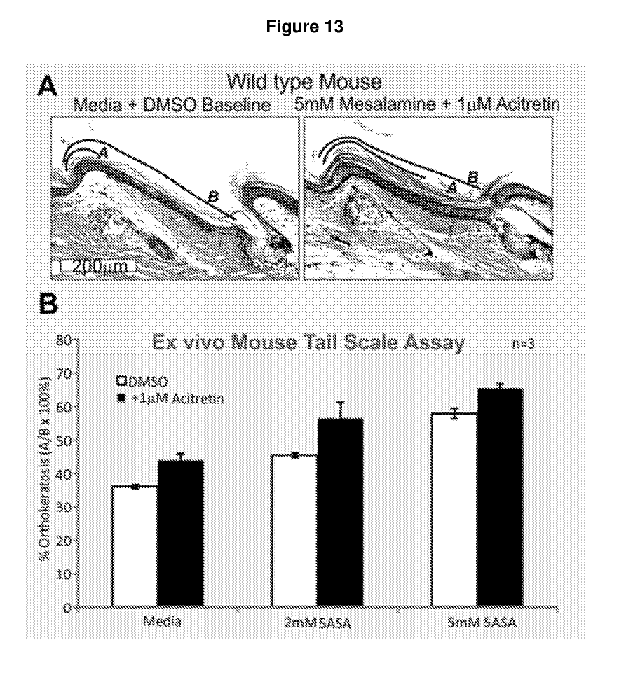

7-9 week old wild type mice of either gender were sacrificed and the tail skin tissue harvested then cultured 4 days on a porous membrane chamber insert with the epidermis exposed to air and dermis side in contact with media drawn through the porous membrane. Various concentrations and combinations of Mesalamine (2 mM and 5 mM) and Acitretin (1 μM) were added to the media or media alone used as a control. Media was refreshed after 48 hrs. Skins were then harvested on the 4 th day and fixed in 4% PFA overnight then stored in 80% Ethanol until processed and embedded in paraffin wax. 8 micron sections were then cut and stained with Haematoxylin and Eosin and analysed. Images were taken from at least 10 scales from 3 mice per treatment condition and the % orthokeratosis measured as defined in the figure legend for FIG. 13.

The mouse tail scale is naturally parakeratotic (ie lacks a granular layer) and has been frequently used as a model system to test drugs for the treatment of psoriasis and other parakeratotic skin diseases, by examining the reacquisition of the granular layer as a measure of orthokeratosis. In this particular assay the treatment with 1 μM Acitretin (the incumbent drug used to treat Harlequin Ichythosis and many other skin diseases) promoted orthokeratosis as expected. Treatment with 2 mM and 5 mM Mesalamine in the culture medium also promoted a dose dependent increase in orthokeratosis, with 2 mM performing similarly to 1 μM Acitretin (FIG. 13; Table 1—mesalamine referred to as 5ASA). This suggests Mesalamine may be a useful alternative to Acitretin in those individuals for whom Acitretin treatment is prohibited. Of particular interest is when Mesalamine and Acitretin where combined they promoted orthokeratosis in an additive manner being the sum of their respective actions (FIG. 13; Table 1). This suggests the two compounds work through different pathways and a combinational formula between Mesalamine and Acitretin may be more effective than an Acitretin treatment alone in parakeratotic diseases such as Harlequin Ichthyosis, dermatitis and psoriasis.

| | t tests p |
|---|---|
| Treatment | values |
| Media v 2 mM 5ASA | 0.001 |
| Media v 5 mM 5ASA | 0.001 |
| 2 mM 5ASA v 5 mM 5ASA | 0.004 |
| 1 uM Acitretin v 2 mM 5ASA + 1 uM Acitretin | 0.071 |
| 1 uM Acitretin v 5 mM 5ASA + 1 uM Acitretin | 0.001 |
| 2 mM 5ASA + 1 uM Acitretin v 5 mM 5ASA + 1 uM Acitretin | 0.121 |
| Media v 1 uM Acitretin | 0.034 |
| 2 mM 5ASA v. 2 mM 5ASA + 1 uM Acitretin | 0.093 |
| 5 mM 5ASA v 5 mM 5ASA + 1 uM Acitretin | 0.016 |
| 2 mM 5ASA v 1 uM Acitretin | 0.294 |

### Example 11

Mesalamine Treatment is More Effective than Acitretin in Lowering Cornified Envelope Thickening in Harlequin Ichthyosis Ex Vivo Embryo Whole Mount Skin Assays.

Embryonic back skin was collected at E16.5 from across 3 litters generated by Abca12 Lx12/+ cross Abca12 Lx12/+ matings to generate Harlequin Ichthyosis embryos Abca12 Lx12/Lx12 and control siblings Abca12 Lx12/+ and Abca12+/+. The embryonic skin was then cultured 4 days on a porous membrane chamber insert with the epidermis exposed to air and dermis side in contact with media drawn through the porous membrane to permit maturation and adaptation to air. 10 mM Mesalamine or 1 uM Acitretin was supplied in the culture media and media refreshed at 48 hrs. Vehicle samples were left without drug treatment. Skins were then harvested on the 4^{th} day and fixed in 4% PFA for 3-4 hrs then stored in 80% Ethanol until processed and embedded in paraffin wax. 8 micron sections were then cut and stained with Haematoxylin and Eosin and analysed. The percentage of skin thickness accounted for by the cornified envelope (cornified layer) was measured and results normalised as a fold change over the vehicle-treated control siblings in each assay. Wild type siblings were under represented in these litters so phenotypically normal heterozygote siblings were used as controls.

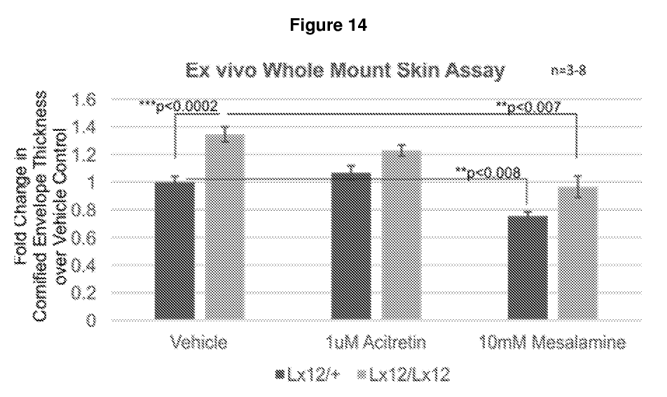

Acitretin is the incumbent retinoid used to treat a variety of skin diseases including HI, although its efficacy in HI is debated and there are serious side effects from long term use. In this assay the inventors set out to compare the action of Mesalamine against Acitretin in the treatment of embryonic HI skin using the ex vivo whole skin culture assay. As expected there was a significant gain in the cornified layer proportion in vehicle-treated Harlequin Ichthyosis (HI) skins, however a strong dose of Acitretin (1 uM) had no significant effect on this measure in HI skins nor control skins. 10 mM Mesalamine on the other hand reduced the cornified layer proportion to vehicle control levels and interestingly also reduced the thickness in control heterozygous Lx12/+ skins (FIG. 14). These results show Mesalamine has greater efficacy in treating HI than Acitretin and may also be beneficial to heterozygous carriers who do not manifest HI but may have an increased incidence of eczema-like sensitivities.

### Example 12

Positive Changes in Epidermal Differentiation Detected Upon Trial of Mesalamine Topical Cream on Live Harlequin Ichthyosis Mice.

Mice of either gender aged 7-9 weeks old and in the telogen phase of the hair cycle had a small area of lower dorsal skin clipped and treated by topical application of 1.5 mg 4-hydroxy-tamoxifen (4OHT) in 100 μl of acetone or acetone vehicle alone. 4OHT was applied every second day for a total of 3 applications over 5 days to induce Harlequin Ichthyosis before application of 200 μl of 2% Mesalamine Cream (or Base cream alone of formula 25% Emu Oil, 22.5% Stearyl Alcohol, 12.5% petroleum jelly, 15% glycerine, 5% Tween 80, 20% water) to back skin and 100 ul of cream applied to tail skin, twice daily at an 8 hr interval for the next 6 days. Mice were sacrificed on day 11 and skin tissue harvested and fixed in 4% PFA in PBS overnight, then stored in 80% Ethanol until processed and wax embedded for paraffin sectioning. 8 μm sections were cut and stained with Haematoxylin and Eosin or DAB-immunostained to detect Keratin 10. Images were acquired using an Aperio brightfield slide scanner and Imagescope software before being analysed using ImageJ software. Blood and urine was collected fresh from sacrificed animals and assayed using a modified trinder assay which utilises a colour-reaction between salicylates and iron and centration of salicylates quantified using spectrometry measurements compared to a known Mesalamine standard dilution series.

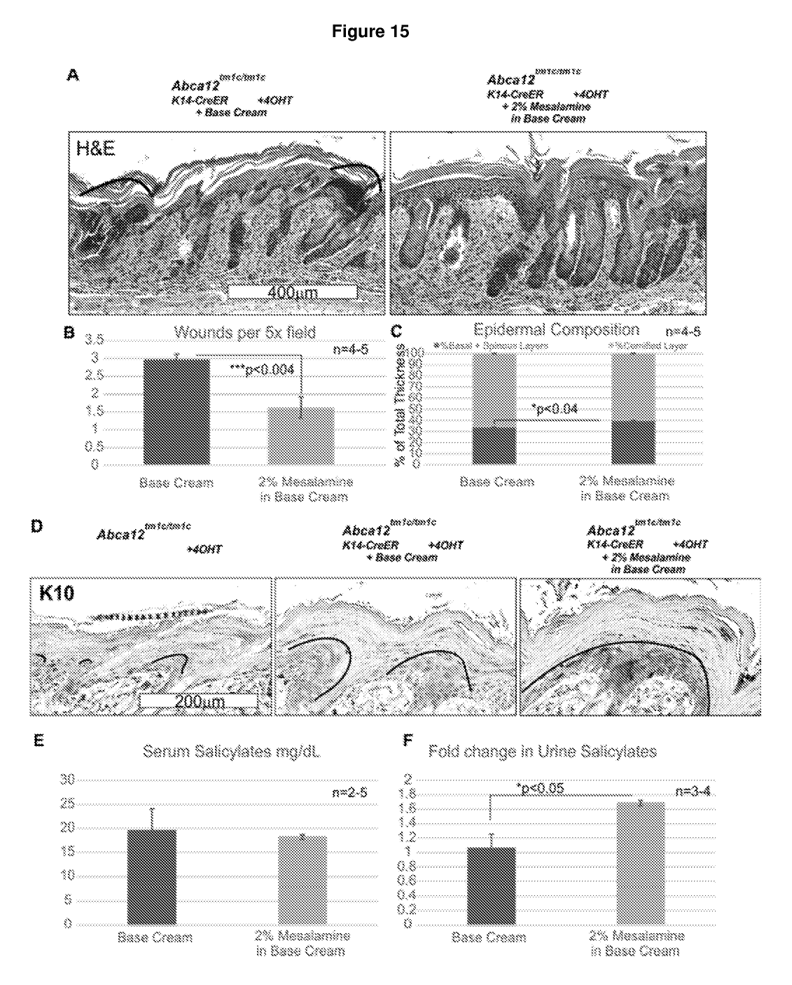

This trial of a topical Mesalamine skin cream occurred on a large live cohort of mice. This first test shows positive changes to the adult mouse Harlequin Ichthyosis skin upon topical Mesalamine treatment including less scabbing, a proportional reduction in the cornified layer thickness, and more robust reacquisition of Keratin 10 than the base cream alone (FIG. 15). This finding on Keratin 10 is especially important, as K10 is an orthokeratotic spinous layer marker whose expression is almost completely abolished in cream-untreated HI mice and its reacquisition demonstrates a direct effect on orthokeratosis. This trial also demonstrated that despite possible systemic absorption of Mesalamine via the skin and from grooming related ingestion, that no salicylate toxicity was observed as Mesalamine was efficiently cleared via urination.

1. A method of treating a skin condition associated with lipid dysfunction, the method comprising administering to a subject in need thereof aminosalicyclic acid (ASA), ASA derivative, or pharmaceutically acceptable salt, ester, amide, polymorph and/or prodrug thereof, thereby treating a skin condition associated with lipid dysfunction.

2. A method according to claim 1, wherein the ASA is mesalamine, 4-ASA or 3-ASA, derivative, or pharmaceutically acceptable salt, ester, amide, polymorph and/or prodrug thereof.

3. A method according to claim 2, wherein the ASA is mesalamine, mesalamine derivative, or pharmaceutically acceptable salt, ester, amide, polymorph and/or prodrug thereof.

4. A method according to any one of claims 1 to 3, wherein the skin condition associated with a lipid dysfunction is an ichthyoses.

5. A method according to claim 4, wherein the ichthyoses is selected from the group consisting of Harlequin Ichthyosis, Lamellar Ichthyosis including various subtypes such as Lamellar Ichthyosis Type 1 or 3, Congenital Ichthyosiform Erythroderma types, Acral Peeling Skin Syndrome, Netherton Syndrome, Chanarin-Dorfman syndrome (Neutral lipid storage disease with Ichthyosis), X-linked Ichthyosis, Arthrogryposis-renal dysfunction-cholestasis (ARC) syndrome, Ichthyosis Vulgaris, Niemann-Pick Disease, Gaucher's Disease and HXALI hepoxilin A3 synthase-linked ichthyosis.

6. A method according to claim 4, wherein the ichthyoses is selected from the group consisting of Harlequin Ichthyosis, Lamellar Ichthyosis including various subtypes such as Lamellar Ichthyosis Type 1 or 3, Congenital Ichthyosiform Erythroderma types, Chanarin-Dorfman syndrome (Neutral lipid storage disease with Ichthyosis), X-linked Ichthyosis, Niemann-Pick Disease, Gaucher's Disease, HXALI hepoxilin A3 synthase-linked ichthyosis.

7. A method according to any one of claims 4 to 6, wherein the ichthyoses is Harlequin Ichthyosis or Lamellar Ichthyosis.

8. A method according to claim 7, wherein the ichthyosis is Harlequin Ichthyosis.

9. A method of alleviating or ameliorating a symptom of a skin condition associated with lipid dysfunction, the method comprising administering to a subject in need thereof ASA, ASA derivative, or pharmaceutically acceptable salt, ester, amide, polymorph and/or prodrug thereof, alleviating or ameliorating a symptom of a skin condition associated with lipid dysfunction.

10. A method according to claim 9, wherein the ASA is mesalamine, 4-ASA or 3-ASA, derivative, or pharmaceutically acceptable salt, ester, amide, polymorph and/or prodrug thereof.

11. A method according to claim 10, wherein the ASA is mesalamine, mesalamine derivative, or pharmaceutically acceptable salt, ester, amide, polymorph and/or prodrug thereof.

12. A method according to any one of claims 1 to 11, wherein the ASA, ASA derivative, or pharmaceutically acceptable salt, ester, amide, polymorph and/or prodrug thereof may be administered directly to the skin.

13. A method according to claim 11, wherein the administration to the skin is via any route that allows ASA, ASA derivative, or pharmaceutically acceptable salt, ester, amide, polymorph and/or prodrug thereof to contact the epidermis or a part thereof.

14. A method according to claim 12, wherein the ASA, ASA derivative, or pharmaceutically acceptable salt, ester, amide, polymorph and/or prodrug thereof may be administered via any route such that it contacts any one of the basal layer, spinous layer, granular layer and stratum corneum.

15. A method according to claim 11, wherein the ASA, ASA derivative, or pharmaceutically acceptable salt, ester, amide, polymorph and/or prodrug thereof is applied to the skin topically.

16. A method according to any one of claims 1 to 15, further comprising administering a retinoid.

17. A method according to claim 16, wherein the retinoid is selected from the group consisting of acitretin, etretinate, isotretinoin and tazarotene.

18. A method according to claim 17, wherein the retinoid is acitretin.

19. A method according to any one of claims 1 to 18, wherein the subject is or has been treated with retinoid therapy.

20. Use of ASA, ASA derivative, or pharmaceutically acceptable salt, ester, amide, polymorph and/or prodrug thereof in the manufacture of a medicament for the treatment of a skin condition associated with lipid dysfunction.

21. Use according to claim 20, wherein the ASA is mesalamine, 4-ASA or 3-ASA, derivative, or pharmaceutically acceptable salt, ester, amide, polymorph and/or prodrug thereof.

22. Use according to claim 20, wherein the ASA is mesalamine, mesalamine derivative, or pharmaceutically acceptable salt, ester, amide, polymorph and/or prodrug thereof.

23. A method for the treatment of a skin condition associated with lipid dysfunction comprising the steps of administering to a subject in need thereof ASA, ASA derivative, or pharmaceutically acceptable salt, ester, amide, polymorph and/or prodrug thereof, and a compound for increasing the barrier function of the skin.

24. A method according to claim 23, wherein the ASA is mesalamine, 4-ASA or 3-ASA, derivative, or pharmaceutically acceptable salt, ester, amide, polymorph and/or prodrug thereof.

25. A method according to claim 23, wherein the ASA is mesalamine, mesalamine derivative, or pharmaceutically acceptable salt, ester, amide, polymorph and/or prodrug thereof.

26. A method according to any one of claims 23 to 25, wherein the compound for increasing the barrier function of the skin creates an artificial skin barrier.

27. A method according to claim 26, wherein the compound is an oil or lipid emollient.

28. A method for the treatment of a skin condition associated with lipid dysfunction comprising the steps of administering a first composition comprising ASA, ASA derivative, or pharmaceutically acceptable salt, ester, amide, polymorph and/or prodrug thereof and a second composition comprising a compound for increasing the barrier function of the skin.

29. A method according to claim 28, wherein the ASA is mesalamine, 4-ASA or 3-ASA, derivative, or pharmaceutically acceptable salt, ester, amide, polymorph and/or prodrug thereof.

30. A method according to claim 28, wherein the ASA is mesalamine, mesalamine derivative, or pharmaceutically acceptable salt, ester, amide, polymorph and/or prodrug thereof.

31. A method according to any one of claims 28 to 30, wherein the first and second compositions are administered sequentially or simultaneously.

32. A method according to any one of claims 28 to 31, wherein the first composition is administered to the subject prior to the second composition.

33. A method according to any one of claims 23 to 32, further comprising administration of a retinoid.

34. A method according to claim 33, wherein the retinoid is selected from the group consisting of acitretin, etretinate, isotretinoin and tazarotene.

35. A method according to claim 34, wherein the retinoid is acitretin.

36. A composition comprising ASA, ASA derivative, or pharmaceutically acceptable salt, ester, amide, polymorph and/or prodrug thereof, for use in the treatment of a skin condition associated with lipid dysfunction.

37. A composition according to claim 36, wherein the ASA is mesalamine, 4-ASA or 3-ASA, derivative, or pharmaceutically acceptable salt, ester, amide, polymorph and/or prodrug thereof.

38. A composition according to claim 36, wherein the ASA is mesalamine, mesalamine derivative, or pharmaceutically acceptable salt, ester, amide, polymorph and/or prodrug thereof.

39. A composition according to any one of claims 36 to 38, further comprising a retinoid.

40. A composition according to claim 39, wherein the retinoid is acitretin.

41. A method of treating dermatitis or psoriasis, the method comprising administering to a subject in need thereof aminosalicyclic acid (ASA), ASA derivative, or pharmaceutically acceptable salt, ester, amide, polymorph and/or prodrug thereof, thereby treating dermatitis or psoriasis.

42. A method according to claim 41, further comprising administering a retinoid.

43. A method according to claim 41, wherein the retinoid is selected from the group consisting of acitretin, etretinate, isotretinoin and tazarotene.

44. A method according to claim 43, wherein the retinoid is acitretin.
